(19) United States
(12) Patent Application Publication
(10) Pub. No.: US 2011/0045408 A1
(43) Pub. Date: Feb. 24, 2011

(54) Color-forming photosensitive composition, lithographic printing plate precursor and novel cyanine dye

(76) Inventors: Shota Suzuki, Shizuoka (JP); Junji Kawaguchi, Shizuoka (JP)

Correspondence Address: BIRCH STEWART KOLASCH & BIRCH, PO BOX 747, FALLS CHURCH, VA, 22040-0747, US

(21) Appl. No.: 12/805,781

(22) Filed: Aug. 19, 2010

(30) Foreign Application Priority Data
Aug. 20, 2009 (JP) P2009-191247

(51) Int. Cl. G03F 7/20; G03F 7/004; C07D 209/04

(52) U.S. Cl. 430/281.1; 430/322; 430/302; 548/455

(57) ABSTRACT

A color-forming photosensitive composition contains: a cyanine dye having at least two polymerizable groups selected from an acryloyl group, a methacryloyl group and a vinyl group; a radical generator; and a monomer having an ethylenically unsaturated group.

### CROSS-REFERENCE TO RELATED APPLICATIONS

This application claims the benefit of Japanese Patent Application JP 2009-191247, filed Aug. 20, 2009, the entire content of which is hereby incorporated by reference, the same as if set forth at length.

### FIELD OF THE INVENTION

The present invention relates to a color-forming photosensitive composition, a lithographic printing plate precursor and a novel cyanine dye. More particularly, it relates to a color-forming photosensitive composition capable of forming color by infrared laser exposure, a lithographic printing plate precursor using the color-forming photosensitive composition and a novel cyanine dye suitable therefor.

### BACKGROUND OF THE INVENTION

In general, a lithographic printing plate is composed of an oleophilic image area accepting ink and a hydrophilic non-image area accepting dampening water in the process of printing. Lithographic printing is a printing method utilizing the nature of water and oily ink to repel with each other and comprising rendering the oleophilic image area of the lithographic printing plate to an ink-receptive area and the hydrophilic non-image area thereof to a dampening water-receptive area (ink-unreceptive area), thereby making a difference in adherence of the ink on the surface of the lithographic printing plate, depositing the ink only to the image area, and then transferring the ink to a printing material, for example, paper.

In order to produce the lithographic printing plate, a lithographic printing plate precursor (PS plate) comprising a hydrophilic support having provided thereon an oleophilic photosensitive resin layer (image-recording layer) is used. Specifically, the PS plate is exposed through a mask, for example, a lith film, and then subjected to development processing, for example, with an alkaline developer to remove the unnecessary image-recording layer corresponding to the non-image area by dissolving while leaving the image-recording layer corresponding to the image area, thereby obtaining the lithographic printing plate.

Due to the recent progress in the technical field, nowadays the lithographic printing plate can be obtained by a CTP (computer-to-plate) technology. Specifically, a lithographic printing plate precursor is directly subjected to scanning exposure using laser or laser diode without using a lith film and developed to obtain a lithographic printing plate.

With the progress described above, the issue on the lithographic printing plate precursor has transferred to improvements, for example, in image-forming property corresponding to the CTP technology, printing property or physical property. Also, with the increasing concern about global environment, as another issue on the lithographic printing plate precursor, an environmental problem on waste liquid discharged accompanying the wet treatment, for example, development processing comes to the front.

In response to the environmental problem, simplification of development or plate making or non-processing has been pursued. As one method of simple plate making, a method referred to as an “on-press development” is practiced. Specifically, according to the method after exposure of a lithographic printing plate precursor, the lithographic printing plate precursor is mounted as it is on a printing machine without conducting conventional development and removal of the unnecessary area of image-recording layer is performed at an early stage of printing step.

Also, as a method of simple development, a method referred to as a “gum development” is practiced wherein the removal of the unnecessary area of image-recording layer is performed using not a conventional high alkaline developer but a finisher or gum solution of near-neutral pH.

In the simplification of plate making operation as described above, a system using a lithographic printing plate precursor capable of being handled in a bright room or under a yellow lump and a light source is preferable from the standpoint of workability. Thus, as the light source, a semiconductor laser emitting an infrared ray having a wavelength of 760 to 1,200 or a solid laser, for example, YAG laser, is used. An UV laser is also used.

As the lithographic printing plate precursor capable of undergoing on-press development, for instance, a lithographic printing plate precursor having provided on a hydrophilic support, an image-recording layer (heat-sensitive layer) containing microcapsules having a polymerizable compound encapsulated therein is described in JP-A-2001-277740 (the term “JP-A” as used herein means an “unexamined published Japanese patent application”) and JP-A-2001-277742. A lithographic printing plate precursor having provided on a support, an image-recording layer (photosensitive layer) containing an infrared absorbing agent, a radical polymerization initiator and a polymerizable compound is described in U.S. Patent Publication No. 2003/0064318. A lithographic printing plate precursor capable of undergoing on-press development having provided on a support, an image-recording layer containing a polymerizable compound and a graft polymer having a polyethylene oxide chain in its side chain or a block polymer having a polyethylene oxide block is described in JP-A-11-277927.

In general, an operation (plate inspection) for inspection and discrimination of image formed on a printing plate is carried out in order to examine whether the image is recorded on the printing plate as intended, in advance of mounting the printing plate on a printing machine. In a conventional lithographic printing plate precursor subjected to a development processing step, since a color image is obtained due to the development processing by means of coloration of the image-recording layer it is ordinarily easily performed to confirm the image formed before the mounting the printing plate on a printing machine.

However, with respect to the lithographic printing plate precursor of the on-press development type or non-processing (non-development) type without accompanying the development processing, the image is not recognized on the printing plate in the step of mounting it on a printing machine, and thus the plate inspection can not be performed. In particular, it is vital in the printing operation to determine whether a registry guide (register mark) which acts as a landmark for the register in multicolor printing is recorded. Therefore, in the lithographic printing plate precursor of the on-press development type or non-processing (non-development) type, a means for confirming the image, that is, color formation or decoloration in the exposed area to form a so-called print-out image is required at the stage of exposure.

From the standpoint of improvement in the workability, it is further required that the print-out image formed by color formation or decoloration maintains the good visibility after the lapse of time.

A lithographic printing plate precursor has been proposed wherein a compound capable of generating an acid, base or radical by means of light or heat and a compound capable of undergoing color change upon interaction with the acid, base or radical generated are used as a print-out agent (for example, see JP-A-11-277927). Also, it has been proposed to utilize color change of thermally decomposable compound as the print-out agent of a direct-drawing type lithographic printing plate precursor having a heat-sensitive layer (for example, see JP-A-2000-335129). Further, it has been proposed to use a thermally decomposable dye having a thermally decomposable temperature of 250° C. or below as the print-out agent (for example, see. JP-A-2003-191657).

According to these proposals, although the color formation or decoloration occurs in the exposed area and the plate inspection property increases to some extent, it is still insufficient.

A system containing an infrared absorbing agent of cyanine dye having a 5-membered ring in its methine chain and a radical generator has good visibility and provides a print-out image at a level capable of performing plate inspection is described in JP-A-2007-90850. However, the technique is still insufficient in view of maintaining the good visibility after the lapse of time.

### SUMMARY OF THE INVENTION

Therefore, an object of the present invention is to provide a color-forming photosensitive composition capable of forming by infrared laser exposure, a color image having good time-lapse stability after the exposure. Another object of the invention is to provide a lithographic printing plate precursor of on-press development type which forms by infrared laser exposure, a color image having good time-lapse stability after the exposure and good plate inspection property and is excellent in printing durability and on-press development property and a plate making method using the lithographic printing plate precursor. A further object of the invention is to provide a cyanine dye capable of forming by infrared laser exposure, a color image having good time-lapse stability after the exposure.

(1) A color-forming photosensitive composition containing (A) a cyanine dye having at least two polymerizable groups selected from an acryloyl group, a methacryloyl group and a vinyl group, (B) a radical generator, and (C) a monomer having an ethylenically unsaturated group.
(2) The color-forming photosensitive composition as described in (1) above, wherein (A) the cyanine dye is a cyanine dye represented by formula (1) shown below.

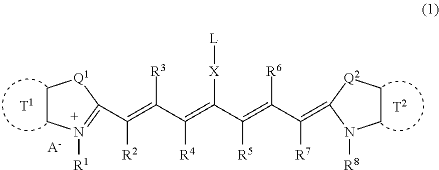

In formula (1), R¹ and R⁸ represent identical organic groups having a polymerizable group selected from an acryloyl group, a methacryloyl group and a vinyl group, R², R³, R⁴, R⁵, R⁶ and R⁷ each independently represents a hydrogen atom or a hydrocarbon group, R⁴ and R⁵ may be combined with each other to form a ring, X represents an oxygen atom, a nitrogen atom or a sulfur atom, L represents an aromatic ring group, a heteroaromatic ring group or an alkyl group having from 1 to 12 carbon atoms, which may contain a hetero atom, provided that when X represents a nitrogen atom, X-L represents —N(L1)(L2) wherein L1 and L2, which may be the same or different, each represents the same group as defined for L, Q¹ and Q², which may be the same or different, each represents —NR⁹—, a sulfur atom, an oxygen atom or a dialkylmethylene group, R⁹ represents a hydrogen atom or a hydrocarbon group which may have a substituent, T¹ and T² each independently represents an aromatic ring or a heteroaromatic ring, wherein the ring may further have a substituent, and A⁻ represents a counter ion.

(3) The color-forming photosensitive composition as described in (1) above, wherein (A) the cyanine dye is a cyanine dye represented by formula (2) shown below.

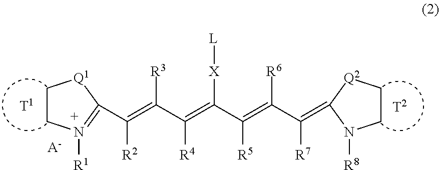

In formula (2), R¹, R², R³, R⁴, R⁵, R⁶, R⁷ and R⁸ each independently represents a hydrogen atom or a hydrocarbon group, R⁴ and R⁵ may be combined with each other to form a ring, X represents an oxygen atom, a nitrogen atom or a sulfur atom, L represents an aromatic ring group, a heteroaromatic ring group or an alkyl group having from 1 to 12 carbon atoms, which may contain a hetero atom, provided that when X represents a nitrogen atom, X-L represents —N(L1)(L2) wherein L1 and L2, which may be the same or different, each represents the same group as defined for L, Q¹ and Q², which may be the same or different, each represents —NR⁹—, a sulfur atom, an oxygen atom or a dialkylmethylene group, R⁹ represents a hydrogen atom or a hydrocarbon group which may have a substituent, T¹ and T² represent the identical aromatic rings or heteroaromatic rings including an organic group having a polymerizable group selected from an acryloyl group, a methacryloyl group and a vinyl group, and A⁻ represents a counter ion.

(4) The color-forming photosensitive composition as described in (1) or (2) above, wherein (A) the cyanine dye is a cyanine dye represented by formula (3) shown below.

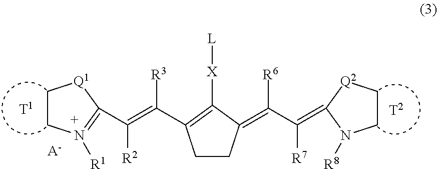

In formula (3), R¹ and R⁸ represent identical organic groups having a polymerizable group selected from an acryloyl group, a methacryloyl group and a vinyl group, R², R³, R⁶ and R⁷ each independently represents a hydrogen atom or a hydrocarbon group, X represents an oxygen atom, a nitrogen atom or a sulfur atom, L represents an aromatic ring group, a heteroaromatic ring group or an alkyl group having from 1 to 12 carbon atoms, which may contain a hetero atom, provided that when X represents a nitrogen atom, X-L represents —N(L1)(L2) wherein L1 and L2, which may be the same or different, each represents the same group as defined for L, Q¹ and Q², which may be the same or different, each represents —NR⁹—, a sulfur atom, an oxygen atom or a dialkylmethylene group, R⁹ represents a hydrogen atom or a hydrocarbon group which may have a substituent, T¹ and T² each independently represents an aromatic ring or a heteroaromatic ring, wherein the ring may further have a substituent, and A⁻ represents a counter ion.

(5) The color-forming photosensitive composition as described in any one of (1) to (4) above, wherein (B) the radical generator is an iodonium salt, a sulfonium salt or an azinium salt.
(6) The color-forming photosensitive composition as described in any one of (1) to (5) above, which further contains (D) a binder polymer.
(7) A lithographic printing plate precursor comprising a support having thereon an image-recording layer containing the color-forming photosensitive composition as described in any one of (1) to (6) above.
(8) The lithographic printing plate precursor as described in (7) above, wherein the image-recording layer further contains (E) a hydrophobilizing precursor.
(9) The lithographic printing plate precursor as described in (7) or (8) above, which further comprises a protective layer on the image-recording layer.
(10) The lithographic printing plate precursor as described in (9) above, wherein the protective layer contains an inorganic stratiform compound.
(11) A method of forming a color image comprising exposing imagewise the color-forming photosensitive composition as described in any one of (1) to (6) above to form color in the exposed area.
(12) A plate making method comprising exposing imagewise the lithographic printing plate precursor as described in any one of (7) to (10) above to form color in the exposed area and mounting the exposed lithographic printing plate precursor on a printing machine or mounting the lithographic printing plate precursor as described in any one of (7) to (10) above on a printing machine and exposing imagewise the lithographic printing plate precursor to form color in the exposed area, and then conducting on-press development processing by supplying printing ink and dampening water.
(13) A cyanine dye represented by formula (4) shown below.

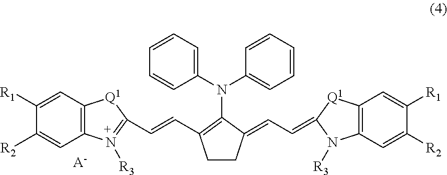

In formula (4), R₁ and R₂ each independently represents a hydrogen atom, a methyl group, a methoxy group or a halogen atom, or R₁ and R₂ may be combined with each other to form a naphtho condensed ring, Q¹ represents a sulfur atom or a dialkylmethylene group, A⁻ represents a halide ion, a perchlorate ion, a tetrafluoroborate ion, a hexafluorophosphate ion or a sulfonate ion, and R₃ represents an organic group having an acryloyl group or a methacryloyl group.

(14) The cyanine dye as described in (13) above, wherein in formula (4), when Q¹ represents a sulfur atom, R₁ represents a hydrogen atom, and R₂ represents a hydrogen atom, a methyl group, a methoxy group or a halogen atom, or when Q¹ represents a dimethylmethylene group, R₂ represents a hydrogen atom, and R₁ represents a hydrogen atom, a methyl group, a methoxy group or a halogen atom, A⁻ represents a hexafluorophosphate ion, and R₃ represents an organic group having an acryloyl group or a methacryloyl group.

According to the invention, the good visibility of color image can be maintained even when time elapsed after the image exposure, by using a specific cyanine dye having at least two polymerizable groups.

The color formation mechanism according to the invention is not quite clear, but it is supposed to be as follows.

It is believed that an efficient electron transfer reaction arises between the cyanine dye (A) excited by infrared laser exposure and a radical generator (B) in the image-recording layer so that polymerization initiation ability generates and also due to the electron transfer the cyanine dye (A) undergoes structural change to cause efficient color change. It is also believed that when the cyanine dye (A) has two or more polymerizable groups, the polymerizable group undergoes a polymerization reaction with a monomer having an ethylenically unsaturated group (C) so that the cyanine dye changed in color is trapped in a polymer matrix to bind its molecular motion and to hardly react with other compounds, whereby the degradation of color image with the lapse of time is prevented.

According to the formation of color image based on the mechanism, it is not preferred to use a coloring agent conventionally used in hitherto known lithographic printing plate precursors because the color change becomes difficult to distinguish.

In JP-A-2004-306582, a lithographic printing plate precursor of on-press development type using a cyanine dye having two allyl groups which belong to a polymerizable group is described. However, there are no descriptions on color formation after image exposure and stability of color image after the lapse of time. Also, in U.S. Patent Publication No. 2007/0269739, a lithographic printing plate precursor of on-press development type using a polymer containing in its side chain a polymerizable group, for example, an acryloyl group and a cyanine dye (infrared absorbing dye) is described. In U.S. Patent Publication No. 2007/0269739, although it is described that in order to obtain a color image after image exposure, a coloring agent which is a derivative of triarylpyridine, xanthene or benzofuran can be added, the technique for obtaining a color image having good stability even afterimage exposure without using a coloring agent as described in the present invention is not described at all.

Therefore, these patent documents neither disclose nor suggest the technique for achieving the formation of good color image and the good stability of image after the lapse of time.

According to the present invention, a color-forming photosensitive composition capable of forming by infrared laser exposure, a color image having good time-lapse stability after the exposure can be provided. Also, according to the invention, a lithographic printing plate precursor of on-press development type which forms by infrared laser exposure, a color image having good time-lapse stability after the exposure and is excellent in printing durability and on-press development property and a plate making method using the lithographic printing plate precursor can be provided. Further, according to the invention, a cyanine dye capable of forming by infrared laser exposure, a color image having good time-lapse stability after the exposure can be provided.

### DETAILED DESCRIPTION OF THE INVENTION

### Color-Forming Photosensitive Composition

The color-forming photosensitive composition according to the invention comprises (A) a dye (hereinafter, also referred to as a specific cyanine dye) having at least two polymerizable groups selected from an acryloyl group, a methacryloyl group and a vinyl group and one cyanine structure, (B) a radical generator, and (C) a monomer having an ethylenically unsaturated group. The composition forms a red-violet to blue-violet color by infrared laser exposure in the exposed area, thereby forming the image having high visibility. The color image formed has high stability and fading and degradation thereof are small with the lapse of time.

The components of the color-forming photosensitive composition according to the invention will be described below.

### (A) Specific Cyanine Dye

The cyanine structure indicates a structure wherein heterocyclic structures each containing a nitrogen atom are connected through a methine chain and a dye having such a structure is called a cyanine dye.

The specific cyanine dye according to the invention is characterized by having at least two polymerizable groups selected from an acryloyl group, a methacryloyl group and a vinyl group and one cyanine structure. Specific examples on the specific cyanine dye include cyanine dyes represented by formulae (1), (2) and (3) shown below.

### <Cyanine Dye Represented by Formula (1)>

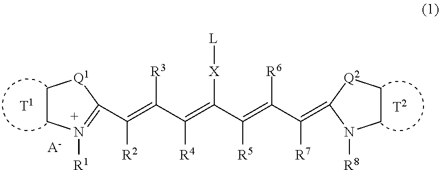

In formula (1), R¹ and R⁸ represent identical organic groups having a polymerizable group selected from an acryloyl group, a methacryloyl group and a vinyl group. The organic group is preferably a group having from 1 to 20 carbon atoms, 10 or less oxygen atoms and 4 or less nitrogen atoms. The acryloyl group or methacryloyl group is preferably connected through an oxygen atom or a nitrogen atom, and more preferably connected through an oxygen atom. The vinyl group is preferably connected through an oxygen atom, an aromatic ring or a hetero ring. Specifically, it is preferably introduced into the cyanine dye, for example, as a vinyloxy group, a vinylphenyl group or a vinylpyridyl group.

Of the polymerizable groups, an acryloyl group or a methacryloyl group is preferred from the standpoint of the plate inspection property and prevention of deterioration of the plate inspection property with the lapse of time.

R², R³, R⁴, R⁵, R⁶ and R⁷ each independently represents a hydrogen atom or a hydrocarbon group. The hydrocarbon group preferably has 12 or less carbon atoms. R⁴ and R⁵ may be combined with each other to form a ring, preferably a 5-membered or 6-membered ring, and more preferably a 5-membered ring.

X represents an oxygen atom, a nitrogen atom or a sulfur atom. L represents an aromatic ring group, preferably an aromatic ring group having 20 or less carbon atoms, a heteroaromatic ring group, preferably a heteroaromatic ring group having 12 or less carbon atoms, or an alkyl group having from 1 to 12 carbon atoms, which may contain a hetero atom. However, when X represents a nitrogen atom, X-L represents —N(L1)(L2) wherein L1 and L2, which may be the same or different, each represents the same group as defined for L. X is preferably a nitrogen atom, and a combination where X is a nitrogen atom and L is an aromatic ring group is more preferred.

Q¹ and Q², which may be the same or different, each represents —NR⁹—, a sulfur atom, an oxygen atom or a dialkylmethylene group, R⁹ represents a hydrogen atom or a hydrocarbon group which may have a substituent. Q¹ and Q² each preferably represents a sulfur atom or a dialkylmethylene group, and more preferably a sulfur atom from the standpoint of the plate inspection property.

T¹ and T² each independently represents an aromatic ring or a heteroaromatic ring, wherein the ring may further have a substituent. T¹ and T² each preferably represents the ring having a benzene structure or a naphthalene structure. It is preferred that the substituent is not present or an electron donating substituent. Preferable examples of the electron donating substituent include an alkyl group or an alkoxy group. A⁻ represents a counter ion, preferably a halide ion, a perchlorate ion, a tetrafluoroborate ion, a hexafluorophosphate ion or a sulfonate ion, and more preferably a hexafluorophosphate ion.

### <Cyanine Dye Represented by Formula (2)>

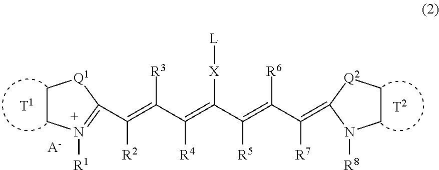

In formula (2), R¹, R², R³, R⁴, R⁵, R⁶, R⁷ and R⁸ each independently represents a hydrogen atom or a hydrocarbon group. The hydrocarbon group preferably has 12 or less carbon atoms. R⁴ and R⁵ may be combined with each other to form a ring, preferably a 5-membered or 6-membered ring, and more preferably a 5-membered ring.

T¹ and T² represent the identical aromatic rings or heteroaromatic rings including an organic group having polymerizable group selected from an acryloyl group, methacryloyl group and a vinyl group. T¹ and T² preferably represent the rings having a benzene structure or a naphthalene structure.

X, L, Q¹, Q², and A⁻ have the same meanings as X, L, Q¹, Q², and A⁻ defined in formula (1) above, respectively.

### <Cyanine Dye Represented by Formula (3)>

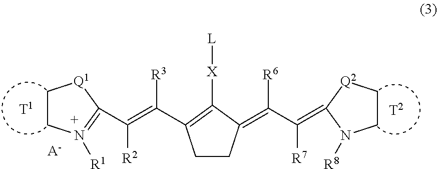

In formula (3), R¹ and R⁸ represent identical organic groups having a polymerizable group selected from an acryloyl group, a methacryloyl group and a vinyl group. The organic group is preferably a group having from 1 to 20 carbon atoms, 10 or less oxygen atoms and 4 or less nitrogen atoms. R², R³, R⁶ and R⁷ each independently represents a hydrogen atom or a hydrocarbon group. The hydrocarbon group preferably has 12 or less carbon atoms. X, L, Q¹, Q², T¹, T² and A⁻ have the same meanings as X, L, Q¹, Q², T¹, T² and A⁻ defined in formula (1) above, respectively.

Of formulae (1) to (3), formulae (1) and (3) wherein the polymerizable groups are introduced into R¹ and R⁸ are preferred, and formulae (3) wherein, in addition, R⁴ and R⁵ are combined with each other to form a 5-membered ring is most preferred.

Specific examples of the specific cyanine dye according to the invention are set forth below, but the invention should not be construed as being limited thereto.

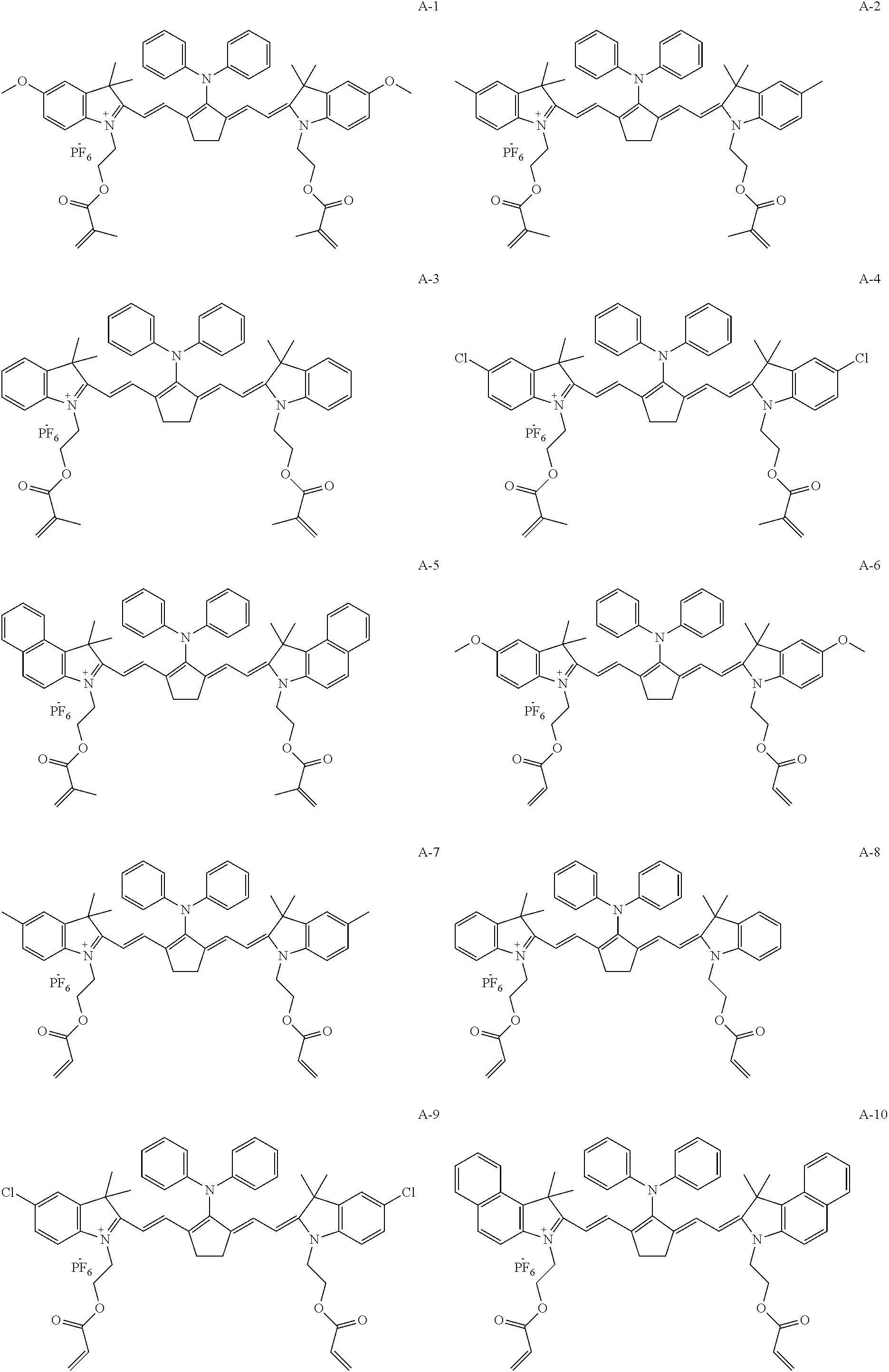

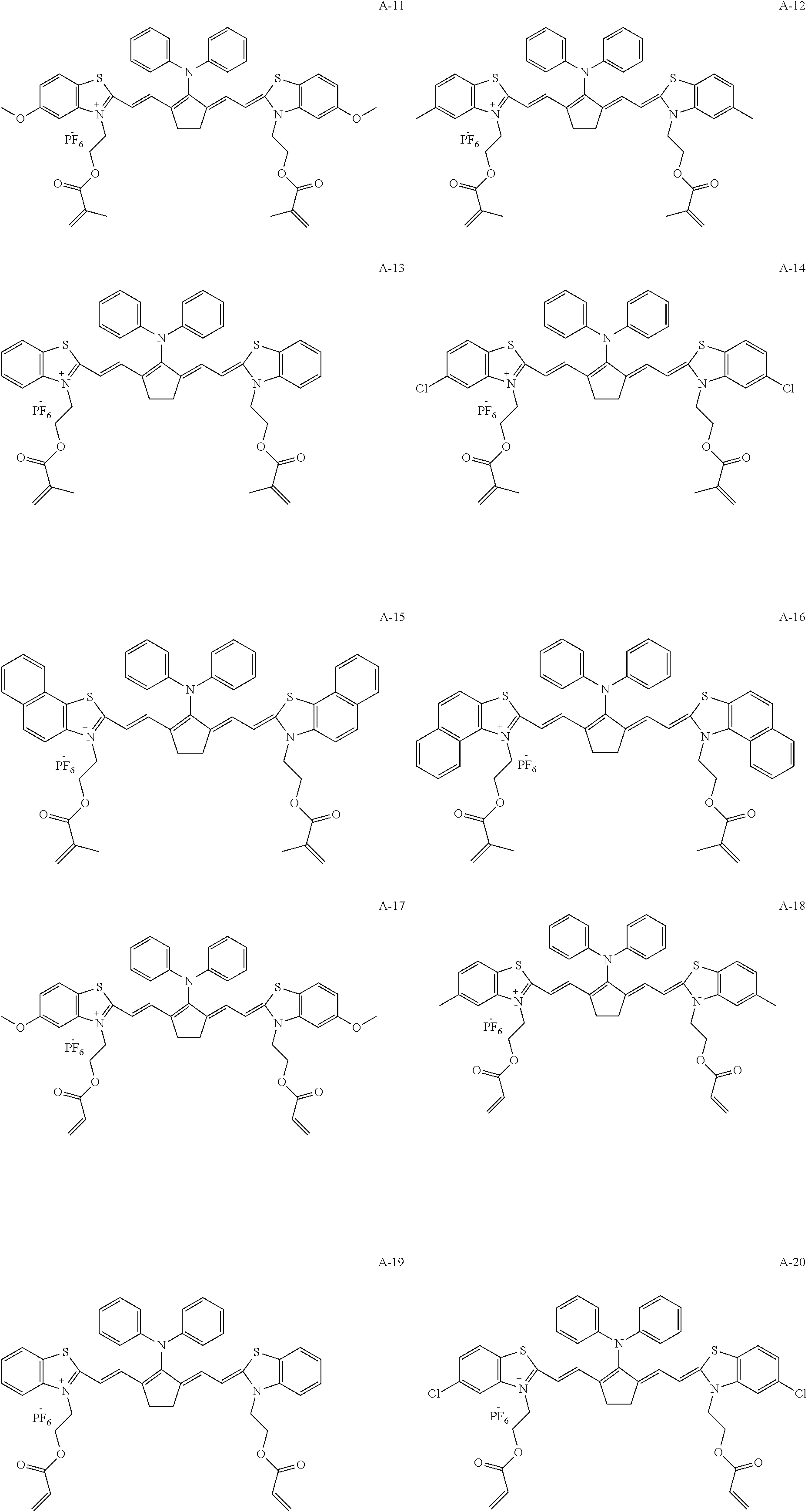

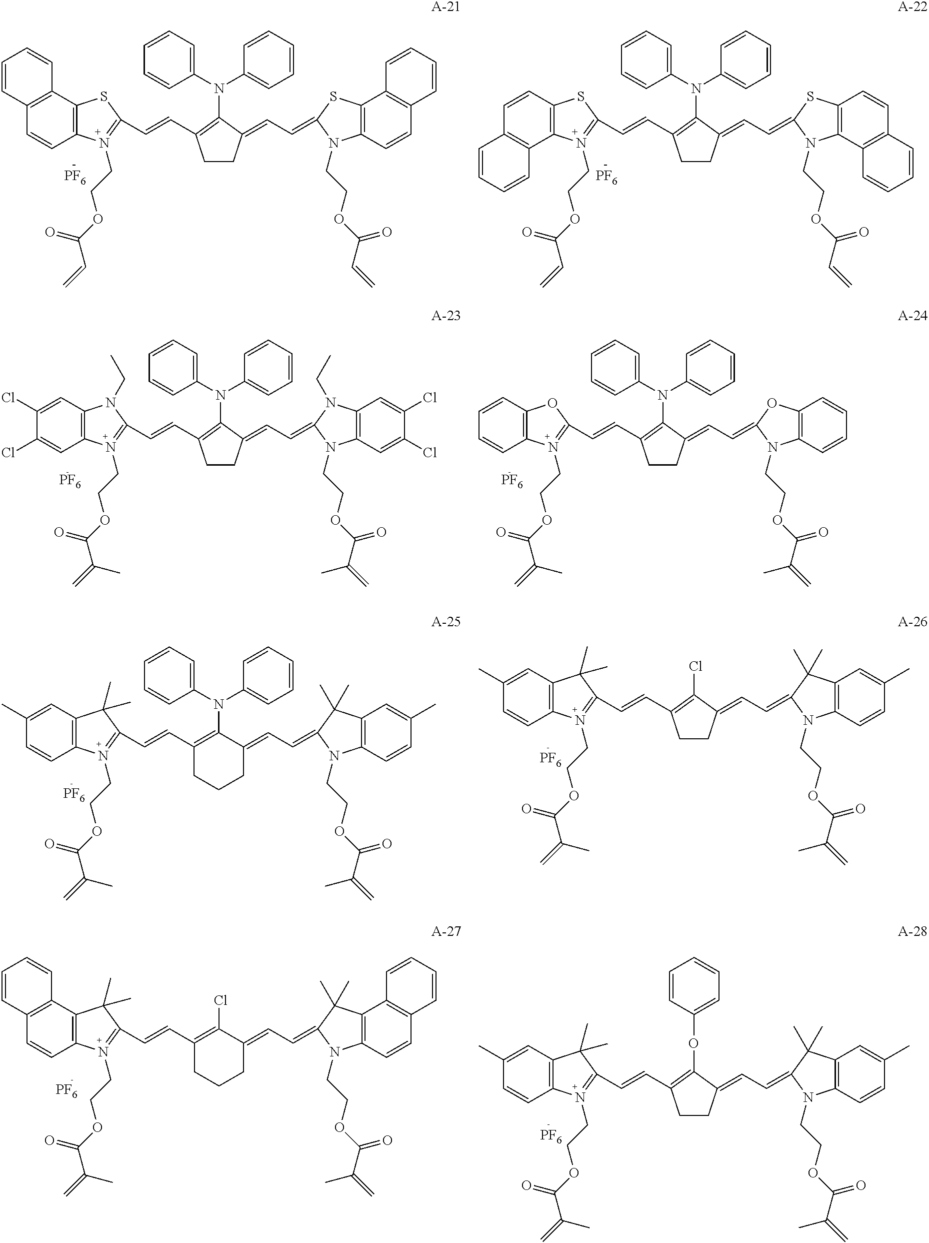

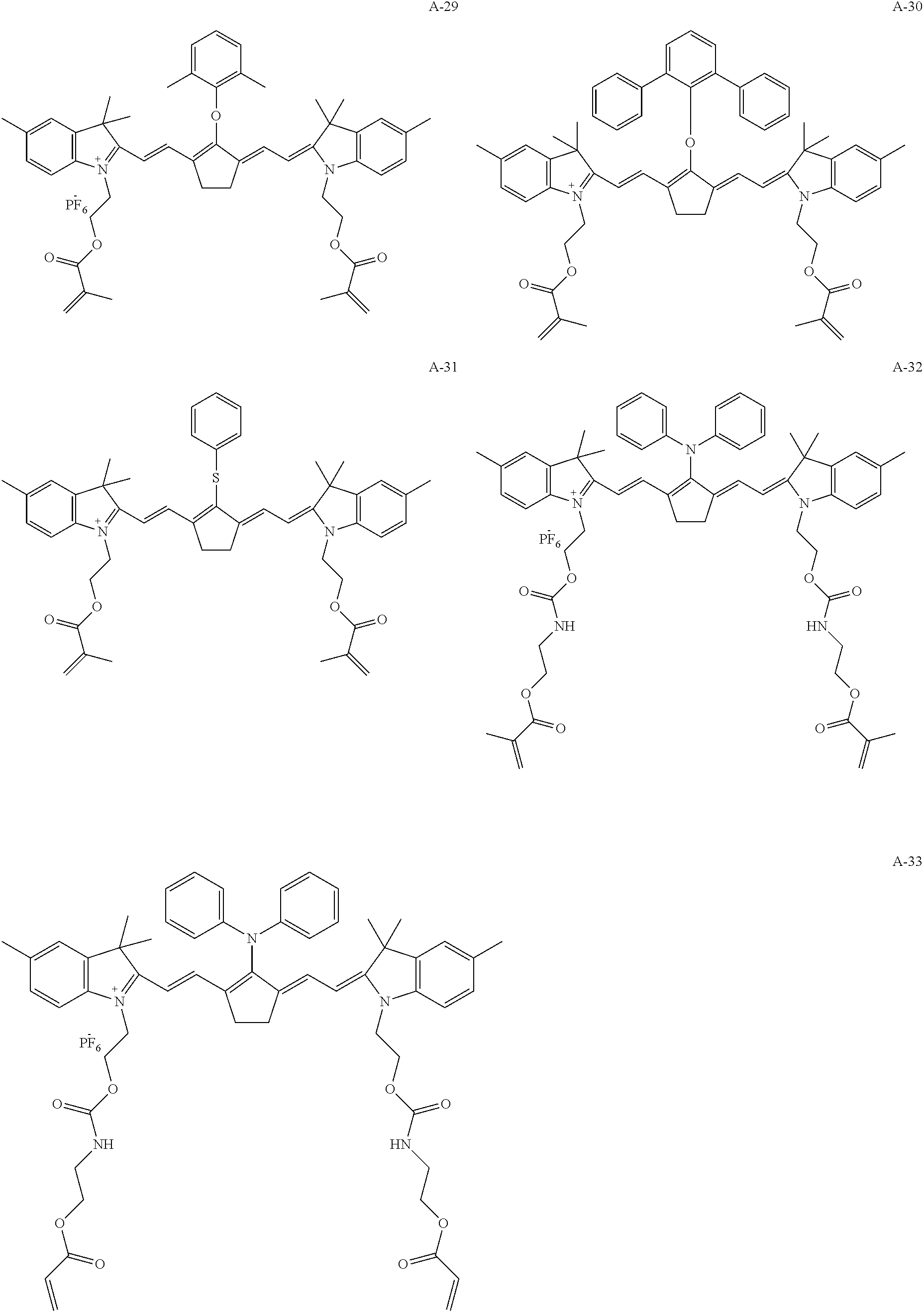

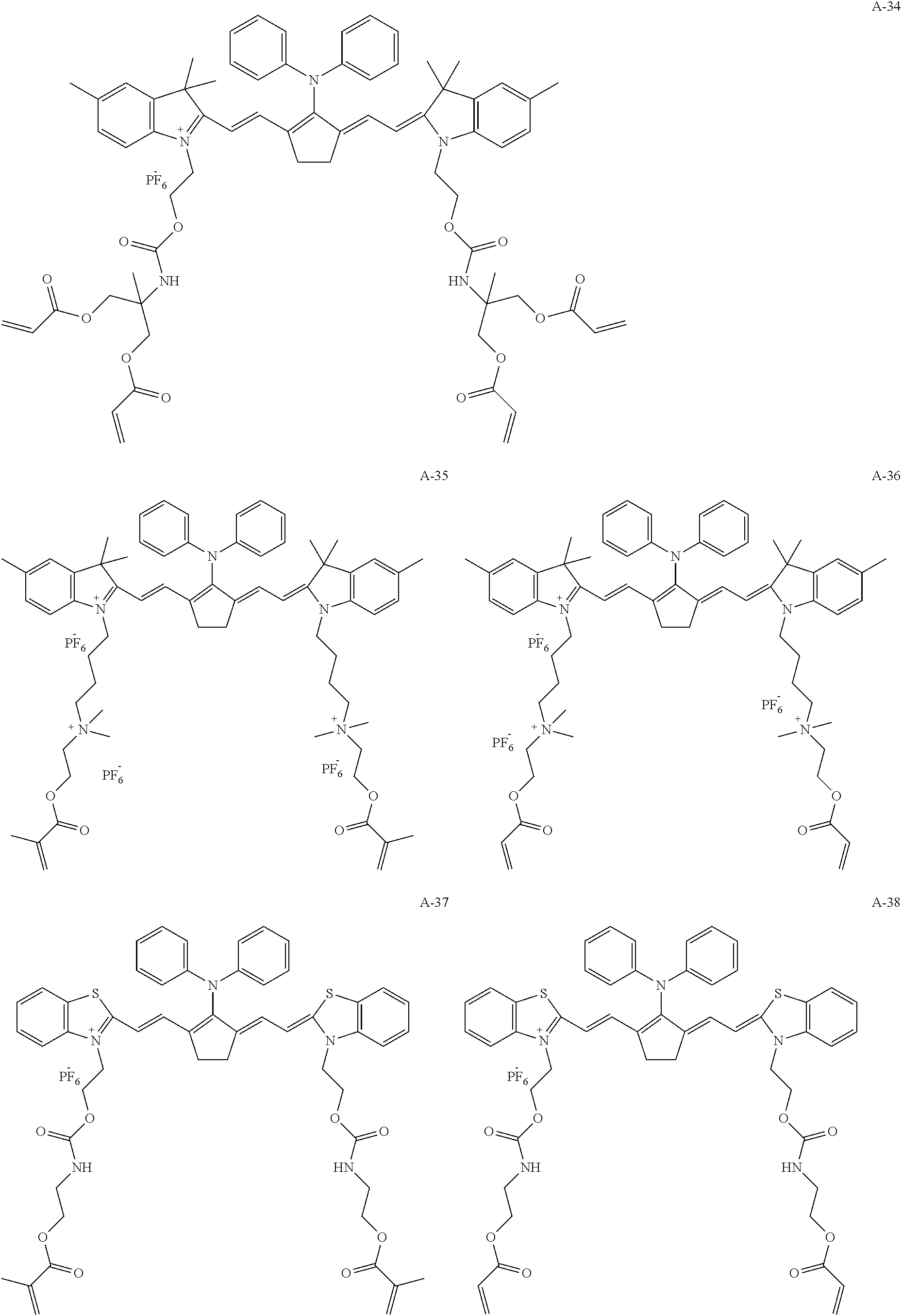

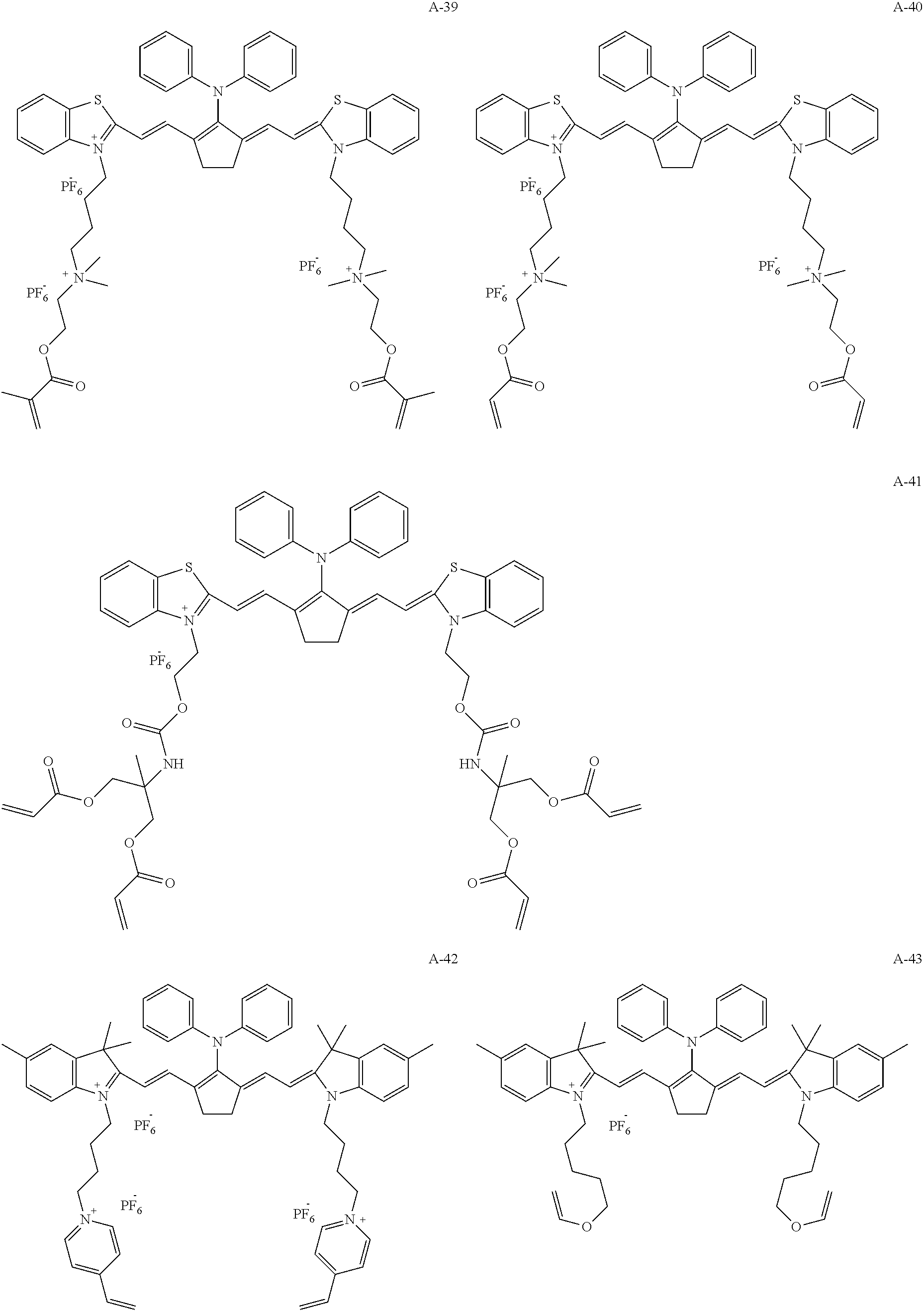

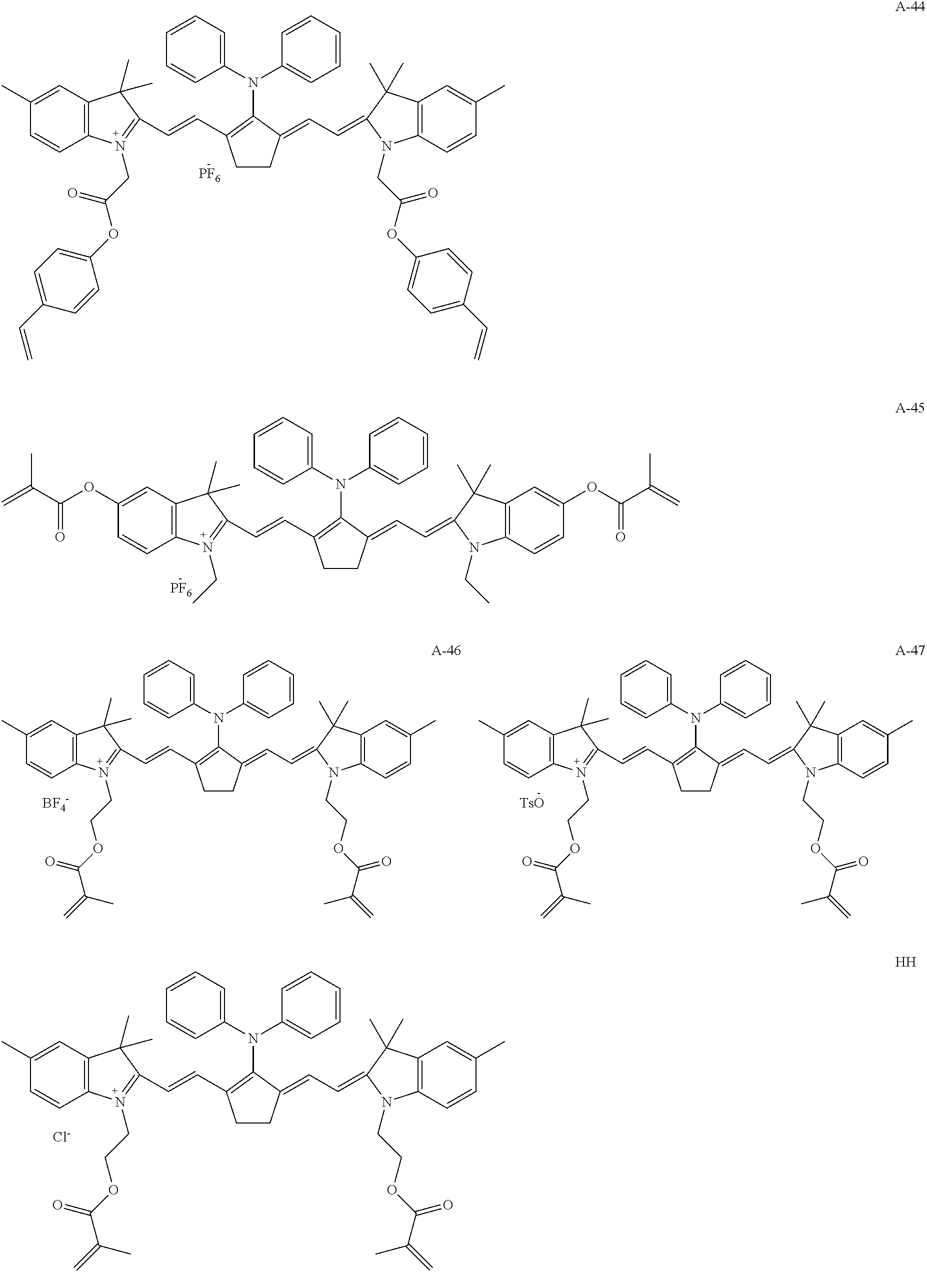

The content of the specific cyanine dye in the color-forming photosensitive composition is preferably from 0.05 to 15.0% by weight, more preferably from 0.1 to 10.0% by weight, most preferably from 0.5 to 5.0% by weight, based on the total solid content constituting the color-forming photosensitive composition. In the range described above, good formation of color and stability of the color image after image exposure are obtained.

### (B) Radical Generator

The radical generator (B) for use in the invention is a compound which initiates or accelerates polymerization of a monomer having an ethylenically unsaturated group (C). The radical generator for use in the invention is preferably a radical polymerization initiator and includes, for example, known thermal polymerization initiators, compounds containing a bond having small bond dissociation energy and photopolymerization initiators.

The radical generators used in the invention include, for example, (a) organic halides, (b) carbonyl compounds, (c) azo compounds, (d) organic peroxides, (e) metallocene compounds, (f) azido compounds, (g) hexaarylbiimidazole compounds, (h) organic borate compounds, (i) disulfone compounds, (j) oxime ester compounds and (k) onium salt compounds.

As the organic halides (a), compounds described in Paragraph Nos. [0022] to [0023] of JP-A-2008-195018 are preferred.

As the carbonyl compounds (b), compounds described in Paragraph No. [0024] of JP-A-2008-195018 are preferred.

As the azo compounds (c), for example, azo compounds described in JP-A-8-108621 are used.

As the organic peroxides (d), for example, compounds described in Paragraph No. [0025] of JP-A-2008-195018 are preferred.

As the metallocene compounds (e), for example, compounds described in Paragraph No. [0026] of JP-A-2008-195018 are preferred.

As the azido compounds (f), compound, for example, 2,6-bis(4-azidobenzylidene)-4-methylcyclohexanone is exemplified.

As the hexaarylbiimidazole compounds (g), for example, compounds described in Paragraph No. [0027] of JP-A-2008-195018 are preferred.

As the organic borate compounds (h), for example, compounds described in Paragraph No. [0028] of JP-A-2008-195018 are preferred.

As the disulfone compounds (i), for example, compounds described in JP-A-61-166544 and JP-A-2002-328465 are exemplified.

As the oxime ester compounds (j), for example, compounds described in Paragraph Nos. [0028] to [0030] of JP-A-2008-195018 are preferred.

As the onium salt compounds (k), onium salts, for example, diazonium salts described in S. I. Schlesinger, Photogr. Sci. Eng., 18, 387 (1974) and T. S. Bal et al., Polymer, 21, 423 (1980), ammonium salts described in U.S. Pat. No. 4,069,055 and JP-A-4-365049, phosphonium salts described in U.S. Pat. Nos. 4,069,055 and 4,069,056, iodonium salts described in European Patent 104,143, U.S. Patent Publication No. 2008/0311520 and JP-A-2-150848 and JP-A-2008-195018, sulfonium salts described in European Patents 370,693, 390,214, 233,567, 297,443 and 297,442, U.S. Pat. Nos. 4,933,377, 4,760,013, 4,734,444 and 2,833,827, German Patents 2,904,626, 3,604,580 and 3,604,581, selenonium salts described in J. V. Crivello et al., Macromolecules, 10 (6), 1307 (1977) and J. V. Crivello et al., J. Polymer Sci., Polymer Chem. Ed., 17, 1047 (1979), arsonium salts described in C. S. Wen et al., Teh, Proc. Conf. Rad. Curing ASIA, p. 478, Tokyo, October (1988), and azinium salts described in JP-A-2008-195018 are exemplified.

Of the radical polymerization initiators, the onium salts, particularly, the iodonium salts, sulfonium salts and azinium salts are preferable. Specific examples of these compounds are set forth below, but the invention should not be construed as being limited thereto.

Of the iodonium salts, diphenyliodonium salts are preferred, diphenyliodonium salts substituted with an electron donating group, for example, an alkyl group or an alkoxy group are more preferred, and asymmetric diphenyliodonium salts are still more preferred. Specific examples thereof include diphenyliodonium hexafluorophosphate, 4-methoxyphenyl-4-(2-methylpropyl)phenyliodonium hexafluorophosphate, 4-(2-methylpropyl)phenyl-p-tolyliodonium hexafluorophosphate, 4-hexyloxyphenyl-2,4,6-trimethoxyphenyliodonium hexafluorophosphate, 4-hexyloxyphenyl-2,4-diethoxyphenyliodoniumtetrafluoroborate, 4-octyloxyphenyl-2,4,6-trimethoxyphenyliodonium 1-perfluorobutanesulfonate, 4-octyloxyphenyl-2,4,6-trimethoxyphenyliodonium hexafluorophosphate and bis(4-tert-butylphenyl)iodonium tetraphenylborate.

Examples of the sulfonium salt include triphenylsulfonium hexafluorophosphate, triphenylsulfonium benzoylformate, bis(4-chlorophenyl)phenylsulfonium benzoylformate, bis(4-chlorophenyl)-4-methylphenylsulfonium tetrafluoroborate and tris(4-chlorophenyl)sulfonium 3,5-bis(methoxycarbonyl)benzenesulfonate.

Examples of the azinium salt include 1-cyclohexylmethyloxypyridinium hexafluorophosphate, 1-cyclohexyloxy-4-phenylpyridinium hexafluorophosphate, 1-ethoxy-4-phenylpyridinium hexafluorophosphate, 1-(2-ethylhexyloxy)-4-phenylpyridinium hexafluorophosphate, 4-chloro-1-cyclohexylmethyloxypyridinium hexafluorophosphate, 1-ethoxy-4-cyanopyridinium hexafluorophosphate, 3,4-dichloro-1-(2-ethylhexyloxy)pyridinium hexafluorophosphate, 1-benzyloxy-4-phenylpyridinium hexafluorophosphate, 1-phenethyloxy-4-phenylpyridinium hexafluorophosphate, 1-(2-ethylhexyloxy)-4-phenylpyridinium p-toluenesulfonate, 1-(2-ethylhexyloxy)-4-phenylpyridinium perfluorobutanesulfonate, 1-(2-ethylhexyloxy)-4-phenylpyridinium bromide and 1-(2-ethylhexyloxy)-4-phenylpyridinium tetrafluoroborate.

The radical generator can be added to the color-forming photosensitive composition preferably in an amount from 0.1 to 50% by weight, more preferably from 0.5 to 30% by weight, particularly preferably from 0.8 to 20% by weight, based on the total solid content constituting the color-forming photosensitive composition. In the range described above, when used in a lithographic printing plate precursor, good sensitivity and good stain resistance in the non-image area at the time of printing are obtained.

### (C) Monomer Having Ethylenically Unsaturated Group

The monomer having an ethylenically unsaturated group (C) for use in the invention is an addition-polymerizable compound which has at least one ethylenically unsaturated double bond and does not have a cyanine structure. Specifically, in the invention, the monomer having an ethylenically unsaturated group (C) is a compound distinguished from the specific cyanine dye (A) described above.

The monomer having an ethylenically unsaturated group (C) is preferably selected from compounds having at least one, preferably two or more, terminal ethylenically unsaturated bonds. Such compounds are widely known in the field of art and they can be used in the invention without any particular limitation. The compound has a chemical form, for example, a monomer, a prepolymer, specifically, a dimer, a trimer or an oligomer, or a (co)polymer thereof, or a mixture thereof.

Specific examples of the monomer having an ethylenically unsaturated group include compounds described in Paragraph Nos. [0089] to [0098] of JP-A-2008-195018. Among them, esters of aliphatic polyhydric alcohol compound with an unsaturated carboxylic acid (for example, acrylic acid, methacrylic acid, itaconic acid, crotonic acid, isocrotonic acid or maleic acid) are preferably exemplified. Other preferable monomers having an ethylenically unsaturated group include monomers having an ethylenically unsaturated group containing an isocyanuric acid structure described in JP-A-2005-329708.

Among them, isocyanuric acid ethylene oxide-modified acrylates, for example, tris(acryloyloxyethyl)isocyanurate or bis(acryloyloxyethyl)hydroxyethyl isocyanurate are particularly preferred because they are excellent in balance between hydrophilicity relating to the on-press development property and polymerization ability relating to the printing durability.

In the invention, the monomer having an ethylenically unsaturated group (C) is preferably used in an amount from 5 to 80% by weight, more preferably from 15 to 75% by weight, based on the total solid content of the color-forming photosensitive composition.

### (D) Binder Polymer

The color-forming photosensitive composition according to the invention may contain a binder polymer for the purpose of improving film strength of a layer formed using the color-forming photosensitive composition. The binder polymer for use in the invention can be selected from those heretofore known for lithographic printing plate precursor without restriction, and polymers having a film-forming property are preferred. Among them, acrylic resins, polyvinyl acetal resins and polyurethane resins are preferred.

As the binder polymer preferable for the invention, a polymer having a crosslinkable functional group for improving film strength of the image area when used in a layer of the color-forming photosensitive composition or lithographic printing plate precursor in its main chain or side chain, preferably in its side chain, as described in JP-A-2008-195018 is exemplified. Due to the crosslinkable functional group, crosslinkage is formed between the polymer molecules to facilitate curing.

As the crosslinkable functional group, an ethylenically unsaturated group, for example, a (meth) acryl group, a vinyl group or an allyl group or an epoxy group is preferable. The crosslinkable functional group can be introduced into the polymer by a polymer reaction or copolymerization. For instance, a reaction between an acrylic polymer or polyurethane having a carboxyl group in its side chain and glycidyl methacrylate or a reaction between a polymer having an epoxy group and a carboxylic acid containing an ethylenically unsaturated group, for example, methacrylic acid can be utilized.

The content of the crosslinkable group in the binder polymer is preferably from 0.1 to 10.0 mmol, more preferably from 1.0 to 7.0 mmol, most preferably from 2.0 to 5.5 mmol, based on 1 g of the binder polymer.

When the binder polymer is used in a photosensitive composition for the lithographic printing plate precursor according to the invention, it is also preferred that the binder polymer further has a hydrophilic group. The hydrophilic group contributes to impart the on-press development property to the image-recording layer. In particular, coexistence of the crosslinkable group and the hydrophilic group makes it possible to maintain good balance between printing durability and developing property.

The hydrophilic group includes, for example, a hydroxy group, a carboxyl group, an alkylene oxide structure, an amino group, an ammonium group, an amido group, a sulfo group and a phosphoric acid group. Among them, an alkylene oxide structure containing from 1 to 9 alkylene oxide units having 2 or 3 carbon atoms is preferable. In order to introduce a hydrophilic group into the binder polymer, a monomer having the hydrophilic group can be copolymerized.

Moreover, an oleophilic group, for example, an alkyl group, an aryl group, an aralkyl group or an alkenyl group may be introduced into the binder polymer according to the invention. Specifically, an oleophilic group-containing monomer, for example, an alkyl methacrylate may be copolymerized. The oleophilic group is useful for controlling ink-receptive property, when the photosensitive composition containing the binder polymer having the oleophilic group is used in the lithographic printing plate precursor.

Specific examples (1) to (13) of the binder polymer for use in the invention are set forth below, but the invention should not be construed as being limited thereto.

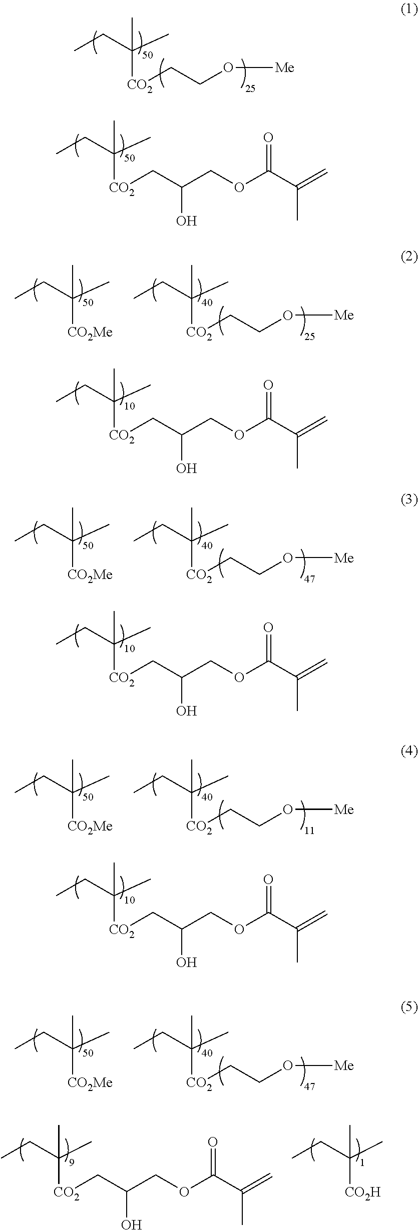

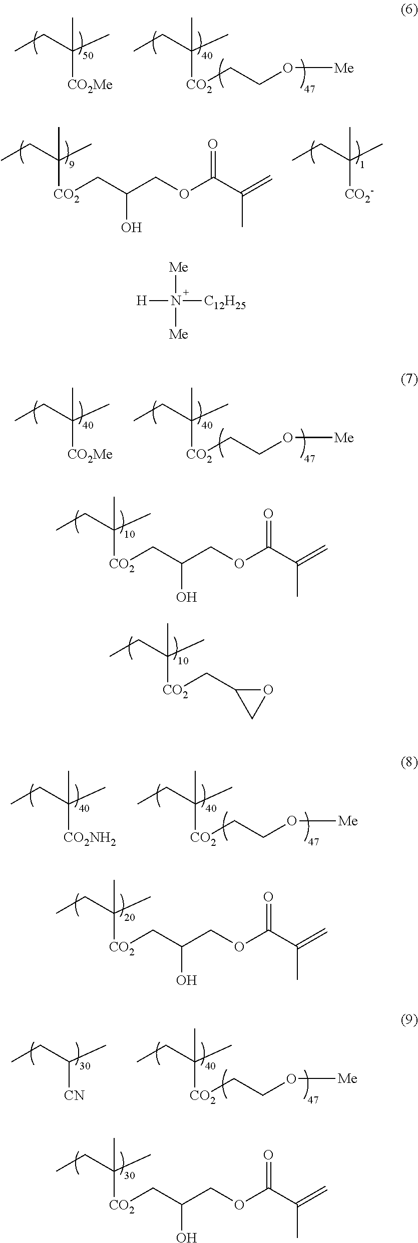

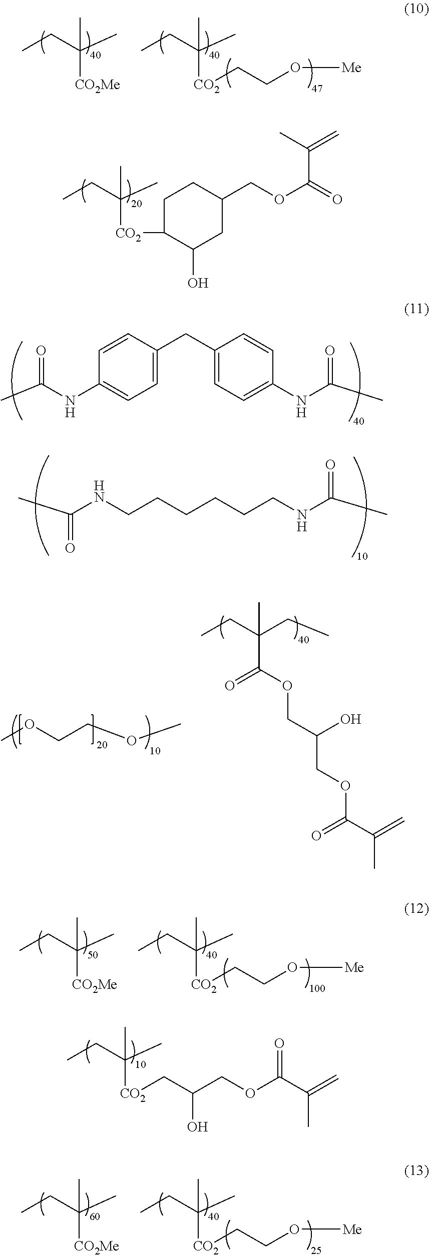

The weight average molecular weight (Mw) of the binder polymer according to the invention is preferably 2,000 or more, more preferably 5,000 or more, and still more preferably from 10,000 to 300,000.

According to the invention, a hydrophilic polymer, for example, polyacrylic acid or polyvinyl alcohol described in JP-A-2008-195018 may be used, if desired. Further, an oleophilic binder polymer is used together with a hydrophilic binder polymer.

The content of the binder polymer is preferably from 5 to 90% by weight, more preferably from 5 to 80% by weight, still more preferably from 10 to 70% by weight, based on the total solid content of the color-forming photosensitive composition.

### (Application of Color-Forming Photosensitive Composition)

The color-forming photosensitive composition may contain additives other than those described above, in accordance with the intended use. The composition is dissolved or dispersed in an appropriate solvent and the resulting solution is coated, for example, on a support and dried to form a layer of the color-forming photosensitive composition, thereby preparing an image-forming material. The image-forming material includes an image-forming material which utilizes color formation and polymerization curing based on imagewise exposure with an infrared ray, for example, a lithographic printing plate precursor, a printed circuit board, a color filter, a photomask or laser color marking. In particular, the color-forming photosensitive composition is preferably used in the production of lithographic printing plate precursor. The infrared ray for use in the invention is preferably an infrared ray laser.

Hereinafter, a lithographic printing plate precursor using the color-forming photosensitive composition described above, particularly a lithographic printing plate precursor of on-press development type will be described in detail.

### [Lithographic Printing Plate Precursor]

The lithographic printing plate precursor according to the invention comprises on a support, an image-recording layer containing the color-forming photosensitive composition described above. The lithographic printing plate precursor according to the invention may have a protective layer on the image-recording layer or an undercoat layer between the support and the image-recording layer. It may further have other layers, if desired.

### (Image-Recording Layer)

The image-recording layer for use in the invention contains the color-forming photosensitive composition described above. Hereinafter, components of the image-recording layer other than the color-forming photosensitive composition described above, method of forming the image-recording layer and the like are described.

### (E) Hydrophobilizing Precursor

According to the invention, a hydrophobilizing precursor can be used in order to improve the on-press development property. The hydrophobilizing precursor for use in the invention is a fine particle capable of converting the image-recording layer to be hydrophobic when heat is applied. The fine particle is preferably at least one fine particle selected from hydrophobic thermoplastic polymer fine particle, thermo-reactive polymer fine particle, microcapsule having a hydrophobic compound encapsulated and microgel (crosslinked polymer fine particle). Among them, polymer fine particle having a polymerizable group and microgel are preferred.

As the hydrophobic thermoplastic polymer fine particle, hydrophobic thermoplastic polymer fine particles described, for example, in Research Disclosure, No. 333003, January (1992), JP-A-9-123387, JP-A-9-131850, JP-A-9-171249, JP-A-9-171250 and European Patent 931,647 are preferably exemplified.

Specific examples of the polymer constituting the polymer fine particle include a homopolymer or copolymer of a monomer, for example, ethylene, styrene, vinyl chloride, methyl acrylate, ethyl acrylate, methyl methacrylate, ethyl methacrylate, vinylidene chloride, acrylonitrile, vinyl carbazole or an acrylate or methacrylate having a polyalkylene structure and a mixture thereof. Among them, polystyrene and polymethyl methacrylate are more preferred.

The average particle size of the hydrophobic thermoplastic polymer fine particle for use in the invention is preferably from 0.01 to 2.0 μm.

The thermo-reactive polymer fine particle for use in the invention includes a polymer fine particle having a thermo-reactive group and forms a hydrophobilized region by crosslinkage due to thermal reaction and change in the functional group involved therein.

As the thermo-reactive group of the polymer fine particle having a thermo-reactive group for use in the invention, a functional group performing any reaction can be used as long as a chemical bond is formed. For instance, an ethylenically unsaturated group (for example, an acryloyl group, a methacryloyl group, a vinyl group or an allyl group) performing a radical polymerization reaction, a cationic polymerizable group (for example, a vinyl group or a vinyloxy group), an isocyanate group performing an addition reaction or a blocked form thereof, an epoxy group, a vinyloxy group and a functional group having an active hydrogen atom (for example, an amino group, a hydroxy group or a carboxyl group) as the reaction partner thereof, a carboxyl group performing a condensation reaction and a hydroxyl group or an amino group as the reaction partner thereof, and an acid anhydride performing a ring opening addition reaction and an amino group or a hydroxyl group as the reaction partner thereof are preferably exemplified.

As the microcapsule for use in the invention, microcapsule having all or part of the constituting components of the image-recording layer encapsulated as described, for example, in JP-A-2001-277740 and JP-A-2001-277742 is exemplified. The constituting components of the image-recording layer may be present outside the microcapsules. It is a more preferable embodiment of the image-recording layer containing microcapsules that hydrophobic constituting components are encapsulated in microcapsules and hydrophilic constituting components are present outside the microcapsules.

The image-recording layer according to the invention is an embodiment containing a crosslinked resin particle, that is, a microgel. The microgel can contain a part of the constituting components of the image-recording layer inside and/or on the surface thereof. Particularly, an embodiment of a reactive microgel containing the monomer having an ethylenically unsaturated group (C) on the surface thereof is preferable from the standpoint of the image-forming sensitivity and printing durability.

As a method of microencapsulation or microgelation of the constituting components of the image-recording layer, known methods can be used.

The average particle size of the microcapsule or microgel is preferably from 0.01 to 3.0 more preferably from 0.05 to 2.0 μm, particularly preferably from 0.10 to 1.0 μm. In the range described above, good resolution and good time-lapse stability can be achieved.

The content of the hydrophobilizing precursor is preferably in a range of 5 to 90% by weight in terms of solid content concentration of the image-recording layer.

### (F) Hydrophilic Low Molecular Weight Compound

The image-recording layer according to the invention may contain a hydrophilic low molecular weight compound in order to improve the on-press development property without accompanying the deterioration of the printing durability.

The hydrophilic low molecular weight compound includes a water-soluble organic compound, for example, a glycol compound, e.g., ethylene glycol, diethylene glycol, triethylene glycol, propylene glycol, dipropylene glycol or tripropylene glycol, or an ether or ester derivative thereof, a polyhydroxy compound, e.g., glycerine, pentaerythritol or tris(2-hydroxyethyl)isocyanurate, an organic amine compound, e.g., triethanolamine, diethanolamine or monoethanolamine, or a salt thereof, an organic sulfonic acid compound, e.g., an alkyl sulfonic acid, toluene sulfonic acid or benzene sulfonic acid, or a salt thereof, an organic sulfamic acid compound, e.g., an alkyl sulfamic acid, or a salt thereof, an organic sulfuric acid compound, e.g., an alkyl sulfuric acid or an alkyl ether sulfuric acid, or a salt thereof, an organic phosphonic acid compound, e.g., phenyl phosphonic acid, or a salt thereof, an organic carboxylic acid, e.g., tartaric acid, oxalic acid, citric acid, malic acid, lactic acid, gluconic acid or an amino acid, or a salt thereof and a betaine compound.

According to the invention, it is preferred that at least one compound selected from a polyol compound, an organic sulfate compound, an organic sulfonate compound and a betaine compound is incorporated.

Specific examples of the organic sulfonate compound include an alkylsulfonate, for example, sodium n-butylsulfonate, sodium n-hexylsulfonate, sodium 2-ethylhexylsulfonate, sodium cyclohexylsulfonate or sodium n-octylsulfonate, an alkylsulfonate containing an ethylene oxide chain, for example, sodium 5,8,11-trioxapentadecane-1-sulfonate, sodium 5,8,11-trioxaheptadecane-1-sulfonate, sodium 13-ethyl-5,8,11-trioxaheptadecane-1-sulfonate or sodium 5,8,11,14-tetraoxatetracosane-1-sulfonate, and an arylsulfonate, for example, sodium benzenesulfonate, sodium p-toluenesulfonate, sodium p-hydroxybenzenesulfonate, sodium p-styrenesulfonate, sodium isophthalic acid dimethyl-5-sulfonate, sodium 1-naphtylsulfonate, sodium 4-hydroxynaphtylsulfonate, disodium 1,5-naphthalenedisulfonate or trisodium 1,3,6-naphthalenetrisulfonate. The salt may also be potassium salt or lithium salt.

The organic sulfate compound includes a sulfate of alkyl, alkenyl, alkynyl, aryl or heterocyclic monoether of polyethylene oxide. The number of unit of ethylene oxide is preferably from 1 to 4. The salt is preferably a sodium salt, a potassium salt or a lithium salt.

As the betaine compound, a compound wherein a number of carbon atoms included in a hydrocarbon substituent on the nitrogen atom is from 1 to 5 is preferred. Specific examples thereof include trimethylammonium acetate, dimethylpropylammonium acetate, 3-hydroxy-4-trimethylammoniobutyrate, 4-(1-pyridinio)butyrate, 1-hydroxyethyl-1-imidazolioacetate, trimethylammonium methanesulfonate, dimethylpropylammonium methanesulfonate, 3-trimethylammonio-1-propanesulfonate and 3-(1-pyridinio)-1-propanesulfonate.

Since the hydrophilic low molecular weight compound has a small structure of hydrophobic portion and almost no surface active function, degradations of the hydrophobicity and film strength in the image area due to penetration of dampening water into the exposed area (image area) of the image-recording layer are prevented and thus, the ink receptive-property and printing durability of the image-recording layer can be preferably maintained.

The amount of the hydrophilic low molecular weight compound added to the image-recording layer is preferably from 0.5 to 20% by weight, more preferably from 1 to 10% by weight, still more preferably from 2 to 8% by weight, based on the total solid content of the image-recording layer. In the range described above, good on-press development property and good printing durability are achieved.

The hydrophilic low molecular weight compounds maybe used individually or as a mixture of two or more thereof.

### (G) Oil-Sensitizing Agent

In order to improve the ink-receptive property, an oil-sensitizing agent, for example, a phosphonium compound, a nitrogen-containing low molecular weight compound or an ammonium group-containing polymer can be used in the image-recording layer. In particular, in the case where an inorganic stratiform compound is incorporated into a protective layer described hereinafter, the oil-sensitizing agent functions as a surface covering agent of the inorganic stratiform compound and prevents deterioration of the ink-receptive property during printing due to the inorganic stratiform compound.

As preferable examples of the phosphonium compound, phosphonium compounds described in JP-A-2006-297907 and JP-A-2007-50660 are exemplified. Specific examples of the phosphonium compound include tetrabutylphosphonium iodide, butyltriphenylphosphonium bromide, tetraphenylphosphonium bromide, 1,4-bis(triphenylphosphonio)butane di(hexafluorophosphate), 1,7-bis(triphenylphosphonio)heptane sulfate and 1,9-bis(triphenylphosphonio)nonane naphthalene-2,7-disulfonate.

As the nitrogen-containing low molecular weight compound, an amine salt and a quaternary ammonium salt are exemplified. Also, an imidazolinium salt, a benzimidazolinium salt, a pyridinium salt and a quinolinium salt are exemplified. Of the nitrogen-containing low molecular weight compounds, the quaternary ammonium salt and pyridinium salt are preferably used. Specific examples the nitrogen-containing low molecular weight compound include tetramethylammonium hexafluorophosphate, tetrabutylammonium hexafluorophosphate, dodecyltrimethylammonium p-toluenesulfonate, benzyltriethylammonium hexafluorophosphate, benzyldimethyloctylammonium hexafluorophosphate and benzyldimethyldodecylammonium hexafluorophosphate.

The ammonium group-containing polymer may be any polymer containing an ammonium group in its structure and is preferably a polymer containing from 5 to 80% by mole of (meth)acrylate having an ammonium group in its side chain as a copolymerization component.

As to the ammonium group-containing polymer, its reduced specific viscosity value (unit: cSt/g/ml) determined according to the measurement method described below is preferably from 5 to 120, more preferably from 10 to 110, particularly preferably from 15 to 100.

### <Measurement Method of Reduced Specific Viscosity>

In a 20 ml measuring flask was weighed 3.33 g of a 30% by weight polymer solution (1 gas a solid content) and the measuring flask was filled up to the gauge line with N-methylpyrrolidone. The resulting solution was put into an Ubbelohde viscometer (viscometer constant: 0.010 cSt/s) and a period for running down of the solution at 30° C. was measured. The viscosity was determined in a conventional manner according to the following calculation formula:

Kinetic viscosity=Viscometer constant×Period for liquid to pass through a capillary (sec)

Specific examples of the ammonium group-containing polymer are set forth below.
(1) 2-(Trimethylammonio)ethyl methacrylate p-toluenesulfonate/3,6-dioxaheptyl methacrylate copolymer (molar ratio: 10/90)
(2) 2-(Trimethylammonio)ethyl methacrylate hexafluorophosphate/3,6-dioxaheptyl methacrylate copolymer (molar ratio: 20/80)
(3) 2-(Ethyldimethylammonio)ethyl methacrylate p-toluenesulfonate/hexyl methacrylate copolymer (molar ratio: 30/70)
(4) 2-(Trimethylammonio)ethyl methacrylate hexafluorophosphate/2-ethylhexyl methacrylate copolymer (molar ratio: 20/80)
(5) 2-(Trimethylammonio)ethyl methacrylate methylsulfate/hexyl methacrylate copolymer (molar ratio: 40/60)
(6) 2-(Butyldimethylammonio)ethyl methacrylate hexafluorophosphate/3,6-dioxaheptyl methacrylate copolymer (molar ratio: 20/80)
(7) 2-(Butyldimethylammonio)ethyl acrylate hexafluorophosphate/3,6-dioxaheptyl methacrylate copolymer (molar ratio: 20/80)
(8) 2-(Butyldimethylammonio)ethyl methacrylate 13-ethyl-5,8,11-trioxa-1-heptadecanesulfonate/3,6-dioxaheptyl methacrylate copolymer (molar ratio: 20/80)
(9) 2-(Butyldimethylammonio)ethyl methacrylate hexafluorophosphate/3,6-dioxaheptyl methacrylate/2-hydroxy-3-methacryloyloxypropyl methacrylate copolymer (molar ratio: 15/80/5)
(10) N-2-(Butyldimethylammonio)ethylmethacrylamide hexafluorophosphate/3,6-dioxaheptyl methacrylate/methacrylamide copolymer (molar ratio: 20/40/40)
(11) 4-(Butyldimethylammoniomethyl)styrene hexafluorophosphate/3,6-dioxaheptyl methacrylate copolymer (molar ratio: 20/80)

The content of the oil-sensitizing agent is preferably from 0.01 to 30.0% by weight, more preferably from 0.1 to 15.0% by weight, still more preferably from 1 to 5% by weight, based on the total solid content of the image-recording layer.

### (H) Other Components

Other components, for example, a surfactant, a print-out agent, a polymerization inhibitor, a higher fatty acid derivative, a plasticizer, a fine inorganic particle, an inorganic stratiform compound, a co-sensitizer or a chain transfer agent may further be added to the image-recording layer. Specifically, compounds and amounts added thereof described, for example, in Paragraph Nos. [0114] to [0159] of JP-A-2008-284817, Paragraph Nos. [0023] to [0027] of JP-A-2006-91479 and Paragraph No. [0060] of U.S. Patent Publication No. 2008/0311520 are preferably used.

### (I) Formation of Image-Recording Layer

The image-recording layer according to the invention is formed by dispersing or dissolving each of the necessary constituting components described above in a known solvent to prepare a coating solution and coating the solution on a support by a known method, for example, bar coater coating and drying as described in Paragraph Nos. [0142] to [0143] of JP-A-2008-195018. The coating amount (solid content) of the image-recording layer formed on a support after coating and drying may be varied according to the intended purpose but is in general preferably from 0.3 to 3.0 g/m². In the range described above, good sensitivity and good film property of the image-recording layer can be achieved.

### (Undercoat Layer)

In the lithographic printing plate precursor according to the invention, an undercoat layer (also referred to as an intermediate layer) is preferably provided between the image-recording layer and the support. The undercoat layer strengthens adhesion between the support and the image-recording layer in the exposed area and makes removal of the image-recording layer from the support in the unexposed area easy, thereby contributing improvement in the developing property without accompanying degradation of the printing durability. Further, it is advantageous that in the case of infrared laser exposure, since the undercoat layer acts as a heat insulating layer, decrease in sensitivity due to diffusion of heat generated upon the exposure into the support is prevented.

As a compound for use in the undercoat layer, specifically, for example, a silane coupling agent having an addition-polymerizable ethylenic double bond reactive group described in JP-A-10-282679 and a phosphorus compound having an ethylenic double bond reactive group described in JP-A-2-304441 are exemplified. A polymer resin having an adsorbing group capable of adsorbing to a surface of the support, a hydrophilic group and a crosslinkable group as described in JP-A-2005-125749 and JP-A-2006-188038 is more preferably exemplified. The polymer resin is preferably a copolymer of a monomer having an adsorbing group, a monomer having a hydrophilic group and a monomer having a crosslinkable group. More specifically, a polymer resin which is a copolymer of a monomer having an adsorbing group, for example, a phenolic hydroxy group, a carboxyl group, —PO₃H₂, —OPO₃H₂, —CONHSO₂—, —SO₂NHSO₂— or —COCH₂COCH₃, a monomer having a hydrophilic sulfo group and a monomer having a polymerizable crosslinkable group, for example, a methacryl group or an allyl group is preferred. The polymer resin may contain a crosslinkable group introduced by a salt formation between a polar substituent of the polymer resin and a compound containing a substituent having a counter charge to the polar substituent of the polymer resin and an ethylenically unsaturated bond and also may be further copolymerized with a monomer other than those described above, preferably a hydrophilic monomer.

The content of the unsaturated double bond in the polymer resin for undercoat layer is preferably from 0.1 to 10.0 mmol, most preferably from 2.0 to 5.5 mmol, based on 1 g of the polymer resin.

The weight average molecular weight of the polymer resin for undercoat layer is preferably 5,000 or more, more preferably from 10,000 to 300,000.

The undercoat layer according to the invention may contain a chelating agent, a secondary or tertiary amine, a polymerization inhibitor or a compound containing an amino group or a functional group having polymerization inhibition ability and a group capable of interacting with the surface of aluminum support (for example, 1,4-diazobicyclo[2,2,2]octane (DABCO), 2,3,5,6-tetrahydroxy-p-quinone, chloranil, sulfophthalic acid, hydroxyethylethylenediaminetriacetic acid, dihydroxyethylethylenediaminediacetic acid or hydroxyethyliminodiacetic acid) in addition to the compounds for the undercoat layer described above in order to prevent the occurrence of stain due to preservation of the lithographic printing plate precursor.

The undercoat layer is coated according to a known method. The coating amount (solid content) of the undercoat layer is preferably from 0.1 to 100 mg/m², and more preferably from 1 to 30 mg/m².

### (Support)

As the support for use in the lithographic printing plate precursor according to the invention, a known support can be used. Particularly, an aluminum plate subjected to roughening treatment and anodizing treatment according to a known method is preferred.

Also, other treatments, for example, an enlarging treatment or a sealing treatment of micropores of the anodized film described in JP-A-2001-253181 and JP-A-2001-322365 or a surface hydrophilizing treatment, for example, with an alkali metal silicate as described in U.S. Pat. Nos. 2,714,066, 3,181,461, 3,280,734 and 3,902,734 or polyvinyl phosphonic acid as described in U.S. Pat. Nos. 3,276,868, 4,153,461 and 4,689,272 may be appropriately selected and applied to the aluminum plate, if desired.

The support preferably has a center line average roughness of 0.10 to 1.2 μm.

The support may have a backcoat layer containing an organic polymer compound described in JP-A-5-45885 or an alkoxy compound of silicon described in JP-A-5-45885, provided on the back surface thereof, if desired.

### (Protective Layer)

As for the lithographic printing plate precursor according to the invention, it is preferred to provide a protective layer (overcoat layer) on the image-recording layer. The protective layer has a function for preventing, for example, occurrence of scratch in the image-recording layer or ablation caused by exposure with a high illuminance laser beam, in addition to the function for restraining an inhibition reaction against the image formation by means of oxygen blocking.

With respect to the protective layer having such properties, there are described, for example, in U.S. Pat. No. 3,458,311 and JP-B-55-49729 (the term “JP-B” as used herein means an “examined Japanese patent publication”). As a polymer having low oxygen permeability for use in the protective layer, any water-soluble polymer and water-insoluble polymer can be appropriately selected to use. Specifically, for example, polyvinyl alcohol, a modified polyvinyl alcohol, polyvinyl pyrrolidone, a water-soluble cellulose derivative and poly(meth)acrylonitrile are exemplified.

It is also preferred that the protective layer contains an inorganic stratiform compound, for example, natural mica or synthetic mica as described in JP-A-2005-119273 in order to increase the oxygen blocking property.

Further, the protective layer may contain a known additive, for example, a plasticizer for imparting flexibility, a surfactant for improving a coating property or a fine inorganic particle for controlling a surface slipping property. The oil-sensitizing agent described with respect to the image-recording layer may also be incorporated into the protective layer.

The protective layer is coated according to a known method. The coating amount of the protective layer is preferably in a range of 0.01 to 10 g/m², more preferably in a range of 0.02 to 3 g/m², most preferably in a range of 0.02 to 1 g/m², in terms of the coating amount after drying.

### [Plate Making Method]

Plate making of the lithographic printing plate precursor according to the invention is preferably performed by an on-press development method. The on-press development method includes a step in which the lithographic printing plate precursor is imagewise exposed and a printing step in which oily ink and an aqueous component are supplied to the exposed lithographic printing plate precursor without undergoing any development processing to perform printing, and it is characterized in that the unexposed area of the image-recording layer and, if present, the protective layer, of the lithographic printing plate precursor are removed in the course of the printing step.

The imagewise exposure may be performed by a platesetter or the like, or in case of using a printing machine equipped with a laser exposure device, the imagewise exposure may be performed on the printing machine after the lithographic printing plate precursor is mounted thereon. In the former case, the exposed lithographic printing plate precursor is mounted as it is on a printing machine without undergoing a development processing step.

The lithographic printing plate precursor according to the invention forms a color image with the specific cyanine dye upon image exposure so that the plate inspection can be conducted using the color image. Then, the printing operation is initiated by supplying oily ink and an aqueous component on the printing machine and at an early stage of the printing the on-press development is carried out. Specifically, the image-recording layer in the unexposed area and, if present, the protective layer, are removed and the hydrophilic surface of support is revealed therewith to form the non-image area. As the oily ink and aqueous component, printing ink and dampening water for conventional lithographic printing can be employed, respectively.

While either the dampening water or printing ink may be supplied at first on the surface of lithographic printing plate precursor, it is preferred to supply the printing ink at first in view of preventing the dampening water from contamination with the component of the image-recording layer removed.

Thus, the lithographic printing plate precursor according to the invention is subjected to the on-press development on an offset printing machine and used as it is for printing a large number of sheets.

The lithographic printing plate precursor according to the invention is able to be subjected to the development processing with a developer using an automatic developing machine, as well as the plate making method including development with dampening water and printing ink on a printing machine. The developer is an aqueous solution preferably having pH of 2 to 11. The developer is preferably an aqueous solution containing water as the main component (containing 60% by weight or more of water). In particular, an aqueous solution containing a surfactant (for example, an anionic, nonionic, cationic or amphoteric surfactant) or an aqueous solution containing a water-soluble polymer compound [for example, gum arabic, dextrin, sterabic, stractan, alginic acid salt, polyacrylic acid salt, hydroxyethyl cellulose, polyvinyl pyrrolidone, polyacrylamide, methyl cellulose, hydroxypropyl cellulose, hydroxymethyl cellulose, carboxyalkyl cellulose salt, water-soluble polysaccharide extracted from soybean curd refuse, pullulan or its derivative or polyvinyl alcohol] is preferred. An aqueous solution containing both the surfactant and the water-soluble polymer compound is also preferred. The pH of the developer is more preferably from 5 to 10.7, still more preferably from 6 to 10.5, and most preferably from 7.5 to 10.3.

The light source used for the image exposure in the invention is preferably a laser. The laser for use in the invention is not particularly restricted and preferably includes, for example, a solid laser or semiconductor laser emitting an infrared ray having a wavelength of 760 to 1,200 nm.

With respect to the infrared ray laser, the output is preferably 100 mW or more, the exposure time per pixel is preferably within 20 microseconds, and the irradiation energy is preferably from 10 to 300 mJ/cm². With respect to the laser exposure, in order to shorten the exposure time, it is preferred to use a multibeam laser device.

### EXAMPLES

The present invention will be described in more detail with reference to the following examples, but the invention should not be construed as being limited thereto.

### Synthesis Example

### Synthesis of Cyanine Dye (A-2)

A mixture of 5.0 g of Compound (A) shown below and 3.61 g of Compound (B) shown below were heated at 120° C. for 8 hours. After cooling to 40° C., 3.54 g of Compound (D), 1.71 g of acetic anhydride, 4.24 g of triethylamine and 30 g of isopropanol were added thereto and the mixture was heated at 80° C. for 10 hours. After cooling the reaction mixture, 500 ml of water was added thereto, followed by stirring for one hour and then the deposit was collected by filtration, washed with water and dried to obtain 4.02 g (yield: 36%) of Intermediate (E). In 20 g of acetone was dissolved 2.0 g of Intermediate (E), 1.21 g of triethylamine and 0.3 mg of p-methoxyphenol were added to the solution, followed by cooling to 0° C. To the reaction solution was added dropwise a solution containing 1.24 g of Compound (F) dissolved in 5 g of acetone over a period of 30 minutes, followed by stirring for one hour. Then, the reaction solution was heated at 60° C. and stirred for 2 days. After cooling to room temperature, 400 ml of water was added thereto, followed by stirring for one hour and then the solid deposited was collected by filtration. The solid collected was dissolved in 100 ml of methanol, the resulting solution was added dropwise to one liter of hexane, followed by stirring for one hour and then the solid deposited was collected by filtration, washed with water and dried to obtain 1.5 g (yield: 64%) of Intermediate (G).

In 100 ml of acetone was dissolved 1.5 g of Intermediate (G), the solution was added dropwise to 300 ml of an aqueous 6% by weight KPF₆ solution, followed by stirring for one hour. The solid deposited was collected by filtration and dried to obtain 1.4 g (yield: 88%) of the desired Cyanine Dye (A-2). The identification of the cyanine dye was conducted by absorption spectrum (solvent: acetonitrile) and ¹H-NMR (solvent: DMSO).

Absorption spectrum: absorption maximum wavelength: 805 nm; molar extinction coefficient (805 nm) : 240,000

¹H-NMR (400 Hz, DMSO-d6): δ 7.48 (d, 2H), 7.44-7.02 (m, 16H), 5.96 (d; 2H), 5.72 (s, 2H), 5.61 (s, 2H), 4.49-4.32 (8H), 2.35 (s, 6H), 1.69 (s, 6H), 1.11-1.05 (m, 12H).

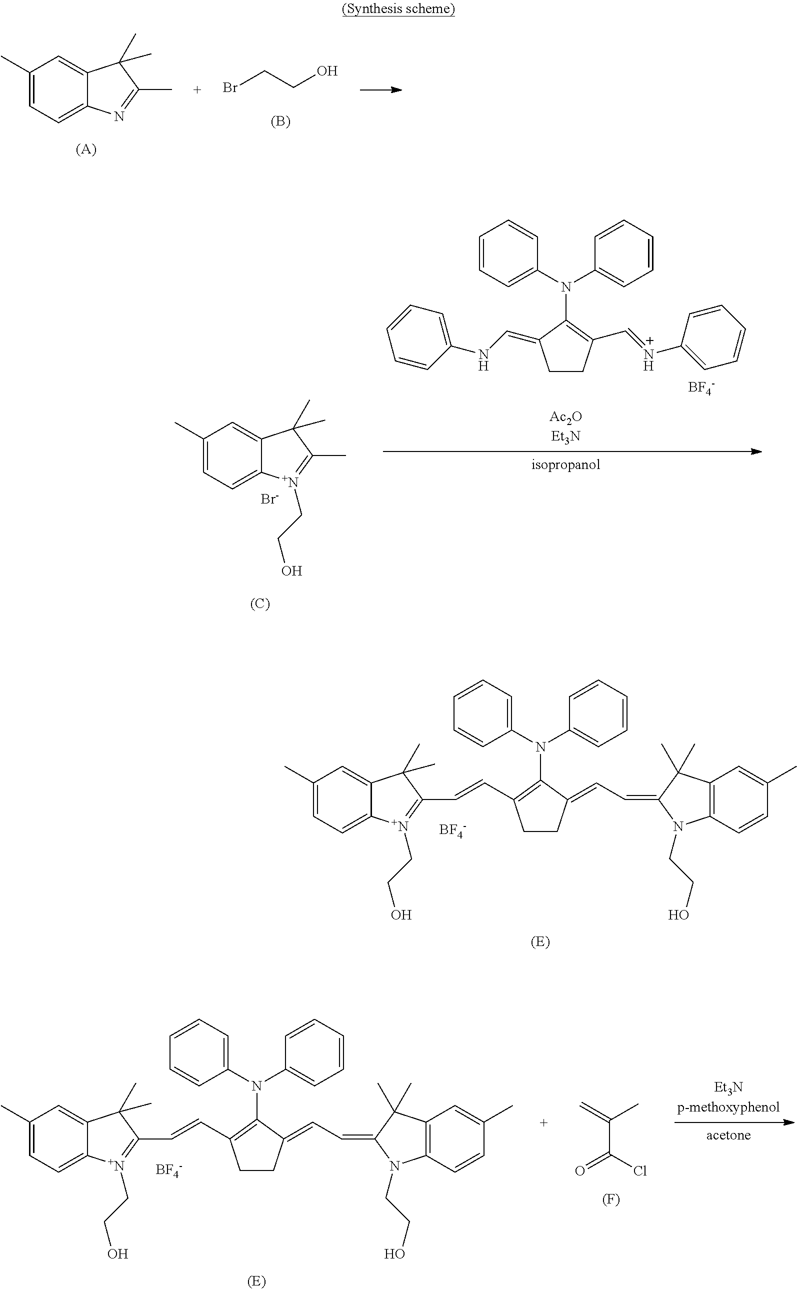

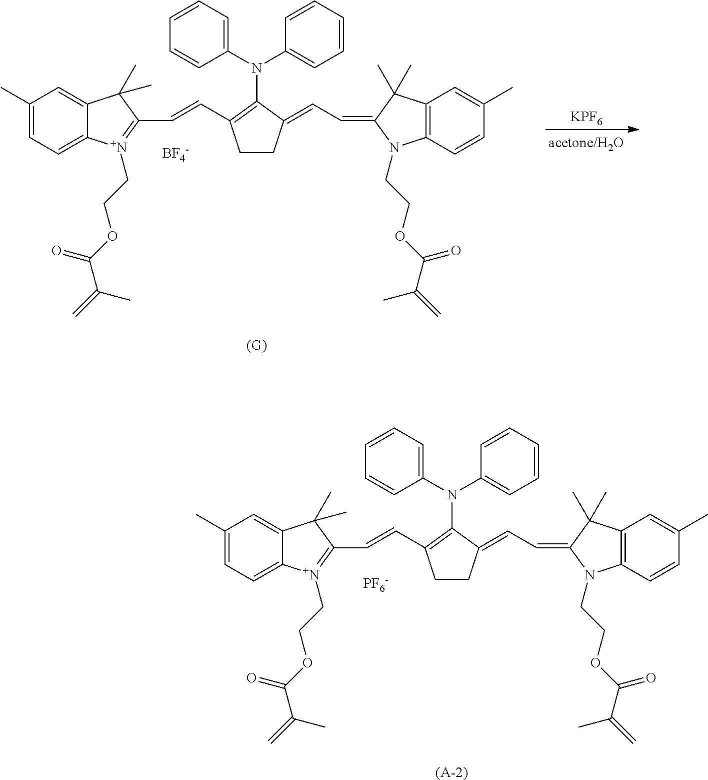

### Example 1 and Comparative Example 1

### Photosensitive Composition

### (1) Preparation of Color-Forming Photosensitive Composition Film 1

Color-forming photosensitive composition solution (1) shown below was prepared and coated on a PET film having a thickness of 0.18 mm subjected to plasma surface treatment using a bar so as to have a dry coating amount of 1.0 g/m² and dried in an oven at 100° C. for 60 seconds to prepare Color-forming photosensitive composition film 1.

### <Color-Forming Photosensitive Composition Solution (1)>

| | |
|---|---|
| Binder polymer (1) having structure shown | 0.636 g |
| below | |
| Specific cyanine dye (A-2) described | 0.030 g |
| hereinbefore | |
| Radical generator (I-1) having structure shown | 0.162 g |
| below | |
| Monomer having ethylenically unsaturated | 0.192 g |
| group (Tris(acryloyloxyethyl) isocyanurate | |
| (NK ESTER A-9300, produced by Shin-Nakamura | |
| Chemical Co., Ltd.)) | |
| Fluorine-based surfactant (1) having | 0.008 g |
| structure shown below | |
| 2-Butanone | 1.091 g |
| 1-Methoxy-2-propanol | 8.609 g |

The structures of Binder polymer (1), Radical generator (I-1) and Fluorine-based surfactant (1) are shown below.

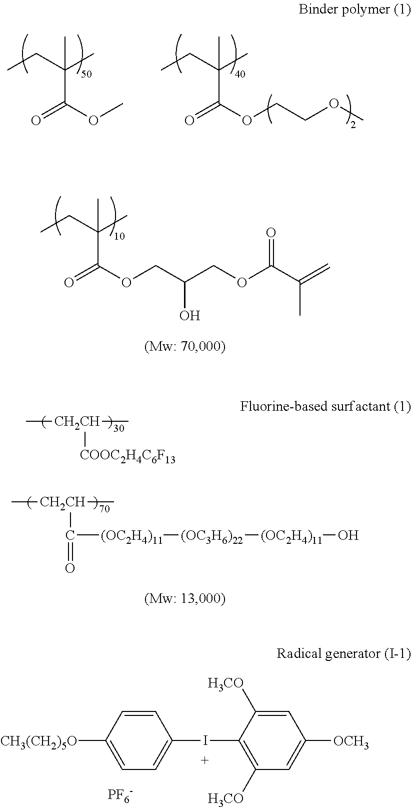

### (2) Preparation of Color-Forming Photosensitive Composition Film 2

Color-forming photosensitive composition film 2 (for Comparative Example 1) was prepared in the same manner as in the preparation of Color-forming photosensitive composition film 1 except for using Cyanine dye (A-49) shown hereinafter in place of Specific cyanine dye (A-2) in Color-forming photosensitive composition solution (1).

### (3) Evaluation of Color-Forming Photosensitive Composition Film

The color-forming photosensitive composition film thus-obtained was exposed by TRENDSETTER 3244VX (produced by Creo Co.) equipped with a water-cooled 40 W infrared semiconductor laser under the conditions of output of 11.7 W, a rotational number of an external drum of 250 rpm and resolution of 2,400 dpi.

Immediately after the exposure and at two hour lapse after the exposure kept under room temperature condition, absorption spectra at 630 nm ware measured and change in the absorption spectrum was determined. The color formation was determined using optical density (OD) and indicated by difference (ΔOD) between the OD value of exposed area and the OD value of unexposed area. When the value of LLD is higher, it is meant that the color-forming property is more excellent. The measurement was conducted by transmission system using Cary 5G UV-Vis-NIR Spectrophotometer produced by Variant Inc. The results obtained are shown in Table 1 below.

TABLE 1
Example 1 and Comparative Example 1

| | Color-Forming | ΔOD | ΔOD |
|---|---|---|---|
| | Photosensitive | (Immediately | (Two Hours |
| | Composition | after | after |
| | Film | Exposure) | Exposure) |
| Example 1 | 1 | 0.35 | 0.25 |
| Comparative | 2 | 0.20 | 0.05 |
| Example 1 | | | |

From the results shown in Table 1, it can be seen that high color formation is obtained and in addition, the high color formation can be kept after the lapse of time when the specific cyanine dye according to the invention is used.

### Examples 2 to 38 and Comparative Examples 2 to 7

### Lithographic Printing Plate Precursor

### 1. Preparation of Lithographic Printing Plate Precursors (1) to (30) and (39) to (42)

### (1) Preparation of Support

An aluminum plate (material: JIS A 1050) having a thickness of 0.3 mm was subjected to a degreasing treatment at 50° C. for 30 seconds using a 10% by weight aqueous sodium aluminate solution in order to remove rolling oil on the surface thereof and then grained the surface thereof using three nylon brushes embedded with bundles of nylon bristle having a diameter of 0.3 mm and an aqueous suspension (specific gravity: 1.1 g/cm³) of pumice having a median size of 25 μm, followed by thorough washing with water. The plate was subjected, to etching by immersing in a 25% by weight aqueous sodium hydroxide solution of 45° C. for 9 seconds, washed with water, then immersed in a 20% by weight aqueous nitric acid solution at 60° C. for 20 seconds, and washed with water. The etching amount of the grained surface was about 3 g/m².

Then, using an alternating current of 60 Hz, an electrochemical roughening treatment was continuously carried out on the plate. The electrolytic solution used was a 1% by weight aqueous nitric acid solution (containing 0.5% by weight of aluminum ion) and the temperature of electrolytic solution was 50° C. The electrochemical roughening treatment was conducted using an alternating current source, which provides a rectangular alternating current having a trapezoidal waveform such that the time TP necessary for the current value to reach the peak from zero was 0.8 msec and the duty ratio was 1:1, and using a carbon electrode as a counter electrode. A ferrite was used as an auxiliary anode. The current density was 30 A/dm² in terms of the peak value of the electric current, and 5% of the electric current flowing from the electric source was divided to the auxiliary anode. The quantity of electricity in the nitric acid electrolysis was 175 C/dm² in terms of the quantity of electricity when the aluminum plate functioned as an anode. The plate was then washed with water by spraying.

The plate was further subjected to an electrochemical roughening treatment in the same manner as in the nitric acid electrolysis above using as an electrolytic solution, a 0.5% by weight aqueous hydrochloric acid solution (containing 0.5% by weight of aluminum ion) having temperature of 50° C. and under the condition that the quantity of electricity was 50 C/dm² in terms of the quantity of electricity when the aluminum plate functioned as an anode. The plate was then washed with water by spraying.

The plate was then subjected to an anodizing treatment using as an electrolytic solution, a 15% by weight aqueous sulfuric acid solution (containing 0.5% by weight of aluminum ion) at a current density of 15 A/dm² to form a direct current anodized film of 2.5 g/m², washed with water and dried to prepare Support (1).

Thereafter, in order to ensure the hydrophilicity of the non-image area, Support (1) was subjected to silicate treatment using a 2.5% by weight aqueous sodium silicate No. 3 solution at 60° C. for 10 seconds and then washed with water to obtain Support (2). The adhesion amount of Si was 10 mg/m². The center line average roughness (Ra) of the support was measured using a stylus having a diameter of 2 μm and found to be 0.51 μm.

(2) Formation of Undercoat layer

Coating solution (1) for undercoat layer shown below was coated on Support (2) so as to have a dry coating amount of 20 mg/m² to prepare a support having an undercoat layer for using the experiments described below.

### <Coating Solution (1) for Undercoat Layer>

| | |
|---|---|
| Compound (1) for undercoat layer | 0.18 g |
| having structure shown below | |
| Hydroxyethyliminodiacetic acid | 0.10 g |
| Methanol | 55.24 g |
| Water | 6.15 g |

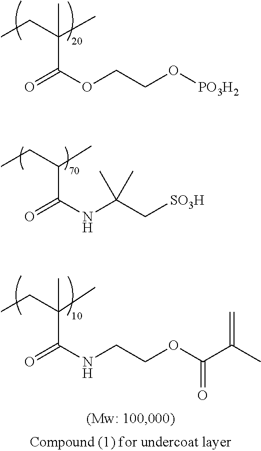

### (3) Formation of Image-Recording Layer

Coating solution (1) for image-recording layer having the composition shown below was coated on the undercoat layer described above by a bar and dried in an oven at 100° C. for 60 seconds to form an image-recording layer having a dry coating amount of 1.0 g/m².

Coating solution (1) for image-recording layer was prepared by mixing Photosensitive solution (1) shown below with Microgel solution (1) shown below just before the coating, followed by stirring.

### <Photosensitive Solution (1)>

| | |
|---|---|
| Binder polymer (1) having structure shown | 0.240 g |
| above | |
| Specific cyanine dye shown in Table 2 | 0.030 g |
| Radical generator shown in Table 2 | 0.162 g |
| Monomer having ethylenically unsaturated | 0.192 g |
| group (Tris(acryloyloxyethyl) isocyanurate | |
| (NK ESTER A-9300, produced by Shin-Nakamura | |
| Chemical Co., Ltd.)) | |
| Hydrophilic low molecular weight compound | 0.062 g |
| (Tris(2-hydroxyethyl) isocyanurate) | |
| Hydrophilic low molecular weight compound (1) | 0.050 g |
| having structure shown below | |
| Oil-sensitizing agent (Phosphonium compound | 0.055 g |
| (1) having structure shown below) | |
| Oil-sensitizing agent (Benzyl dimethyl octyl | 0.018 g |
| ammonium PF₆ salt | |
| Oil-sensitizing agent (Ammonium | 0.035 g |
| group-containing polymer having structure | |
| shown below (reduced specific viscosity: 44 | |
| cSt/g/ml) | |
| Fluorine-based surfactant (1) having | 0.008 g |
| structure shown above | |
| 2-Butanone | 1.091 g |
| 1-Methoxy-2-propanol | 8.609 g |

### <Microgel Solution (1)>

| | |
|---|---|
| Microgel (1) shown below | 2.640 g |
| Distilled water | 2.425 g |

The structures of Hydrophilic low molecular weight compound (1), Phosphonium compound (1) and ammonium group-containing polymer are shown below.

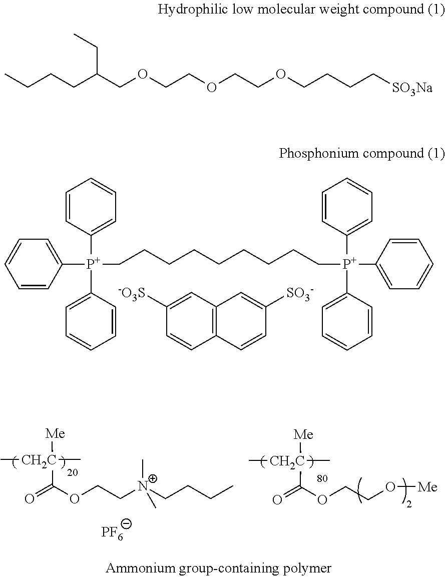

### <Preparation of Microgel (1)>

An oil phase component was prepared by dissolving 10 g of adduct of trimethylol propane and xylene diisocyanate (TAKENATE D-110N, produced by Mitsui Chemicals Polyurethanes, Inc.), 3.15 g of pentaerythritol triacrylate (SR444, produced by Nippon Kayaku Co., Ltd.) and 0.1 g of PIONIN A-41C (produced by Takemoto Oil & Fat Co., Ltd.) in 17 g of ethyl acetate. As an aqueous phase component, 40 g of a 4% by weight aqueous solution of polyvinyl alcohol (PVA-205 produced by Kuraray Co., Ltd.) was prepared. The oil phase component and the aqueous phase component were mixed and emulsified using a homogenizer at 12,000 rpm for 10 minutes. The resulting emulsion was added to 25 g of distilled water and stirred at room temperature for 30 minutes and then at 50° C. for 3 hours. The microgel liquid thus-obtained was diluted using distilled water so as to have the solid concentration of 15% by weight to prepare Microgel (1). The average particle size of the microgel was measured by a light scattering method and found to be 0.2 μm.

### (4) Formation of Protective Layer

Coating solution (1) for protective layer having the composition shown below was coated on the image-recording layer described above by a bar and dried in an oven at 120° C. for 60 seconds to form a protective layer having a dry coating amount of 0.15 g/m², thereby preparing Lithographic printing plate precursors (1) to (30) for Examples 2 to 31 and Lithographic printing plate precursors (39) to (42) for Comparative Examples 2 to 5, respectively.

### <Coating Solution (1) for Protective Layer>

| | | |
|---|---|---|
| Dispersion of inorganic stratiform compound | 1.5 | g |
| (1) shown below | | |
| Aqueous 6% by weight solution of polyvinyl | 0.55 | g |
| alcohol (CKS 50, sulfonic acid-modified, | | |
| saponification degree: 99% by mole or more, | | |
| polymerization degree: 300, produced by Nippon | | |
| Synthetic Chemical Industry Co., Ltd.) | | |
| Aqueous 6% by weight solution of polyvinyl | 0.03 | g |
| alcohol (PVA-405, saponification degree: | | |
| 81.5% by mole, polymerization degree: 500, | | |
| produced by Kuraray Co., Ltd.) | | |
| Aqueous 1% by weight solution of surfactant | 0.86 | g |
| (EMALEX 710, produced by Nihon Emulsion Co., | | |
| Ltd.) | | |
| Ion-exchanged water | 6.0 | g |

### <Preparation of Dispersion of Inorganic Stratiform Compound (1)>

To 193.6 g of ion-exchanged water was added 6.4 g of synthetic mica (SOMASIF ME-100, produced by CO—OP Chemical Co., Ltd.) and the mixture was dispersed using a homogenizer until an average particle size (according to a laser scattering method) became 3 μm to prepare Dispersion of inorganic stratiform compound (1). The aspect ratio of the inorganic particle thus-dispersed was 100 or more.

### 2. Preparation of Lithographic Printing Plate Precursors (37) and (43)

Lithographic printing plate precursor (37) for Example 38 and Lithographic printing plate precursor (43) for Comparative Example 6 were prepared in the same manner as in Lithographic printing plate precursors (2) and (40) respectively except for using Coating solution (2) for protective layer which did not contain Dispersion of inorganic stratiform compound (1) in Coating solution (1) for protective layer in place of Coating solution (1) for protective layer.

### 3. Preparation of Lithographic Printing Plate Precursors (31) to (33) and (44)

Coating solution (2) for image-recording layer having the composition shown below was coated on the support provided with the undercoat layer described above by a bar and dried in an oven at 70° C. for 60 seconds to form an image-recording layer having a dry coating amount of 0.6 g/m², thereby preparing Lithographic printing plate precursors (31) to (33) for Examples 32 to 34 and Lithographic printing plate precursor (44) for Comparative Example 7, respectively.

### <Coating Solution (2) for Image-Recording Layer>

| | | |
|---|---|---|
| Aqueous dispersion of fine polymer particle | 20.0 | g |
| (1) prepared as shown below | | |
| Specific cyanine dye shown in Table 3 | 0.2 | g |
| Radical generator (IRGACURE 250, produced by | 0.5 | g |
| Ciba Specialty Chemicals, Inc.) | | |
| Monomer having ethylenically unsaturated | 1.50 | g |
| group (SR-399, produced by Sartomer Co.) | | |
| Mercapto-3-triazole | 0.2 | g |
| BYK 336 (produced by BYK-Chemie GmbH) | 0.4 | g |
| KLUCEL M (produced by Hercules Chemical Co., | 4.8 | g |
| Inc.) | | |
| ELVACITE 4026 (produced by Ineos Acrylics | 2.5 | g |
| Inc.) | | |
| n-Propanol | 55.0 | g |
| 2-Butanone | 17.0 | g |

The compounds indicated using their trade names in the composition above are shown below.

IRGACURE 250: (4-Methoxyphenyl)[4-(2-methylpropyl)phenyl]iodonium hexafluorophosphate (75% by weight propylene carbonate solution)
SR-399: Dipentaerythritol pentaacrylate
BYK 336: Modified dimethylpolysiloxane copolymer (25% by weight xylene/methoxypropyl acetate solution)
KLUCEL M: Hydroxypropyl cellulose (2% by weight aqueous solution)
ELVACITE 4026: Highly branched polymethyl methacrylate (10% by weight 2-butanone solution)

### (Preparation of Aqueous Dispersion of Fine Polymer Particle (1))

A stirrer, a thermometer, a dropping funnel, a nitrogen inlet tube and a reflux condenser were attached to a 1,000 ml four-neck flask and while carrying out deoxygenation by introduction of nitrogen gas, 10 g of polyethylene glycol methyl ether methacrylate (PEGMA, average number of ethylene glycol repeating units: 50), 200 g of distilled water and 200 g of n-propanol were charged therein and heated until the internal temperature reached 70° C. Then, a mixture of 10 g of styrene (St), g of acrylonitrile (AN) and 0.8 g of 2,2′-azobisisobutyronitrile previously prepared was dropwise added to the flask over a period of one hour. After the completion of the dropwise addition, the reaction was continued as it was for 5 hours. Then, 0.4 g of 2,2′-azobisisobutyronitrile was added and the internal temperature was raised to 80° C. Thereafter, 0.5 g of 2,2′-azobisisobutyronitrile was added over a period of 6 hours. At the stage after reacting for 20 hours in total, the polymerization proceeded 98% or more to obtain Aqueous dispersion of fine polymer particle (1) of PEGMA/St/AN (10/10/80 in a weight ratio). The particle size distribution of the fine polymer particle had the maximum value at the particle size of 150 nm.

The particle size distribution was determined by taking an electron microphotograph of the fine polymer particle, measuring particle sizes of 5,000 fine particles in total on the photograph, and dividing a range from the largest value of the particle size measured to 0 on a logarithmic scale into 50 parts to obtain occurrence frequency of each particle size by plotting. With respect to the aspherical particle, a particle size of a spherical particle having a particle area equivalent to the particle area of the aspherical particle on the photograph was defined as the particle size.

### 4. Preparation of Lithographic Printing Plate Precursors (34) to (36)

Coating solution (3) for image-recording layer having the composition shown below was coated on the support provided with the undercoat layer described above by a bar and dried in an oven at 70° C. for 60 seconds to form an image-recording layer having a dry coating amount of 0.6 g/m².

### <Coating Solution (3) for Image-Recording Layer>

| | | |
|---|---|---|
| Aqueous dispersion of fine polymer particle (2) | 33.0 | g |
| prepared as shown below | | |
| Specific cyanine dye shown in Table 3 | 1.0 | g |
| Radical generator shown in Table 3 | 0.6 | g |
| Monomer having ethylenically unsaturated group | 0.7 | g |
| (Tris (acryloyloxyethyl) isocyanurate (NK ESTER | | |
| A-9300, produced by Shin-Nakamura Chemical Co., | | |
| Ltd.)) | | |
| Polyacrylic acid (weight average molecular | 0.4 | g |
| weight: 20,000) | | |
| Disodium 1,5-naphthalenedisulfonate | 0.1 | g |
| Methanol | 16.0 | g |

### (Preparation of Aqueous Dispersion of Fine Polymer Particle (2))

A stirrer, a thermometer, a dropping funnel, a nitrogen inlet tube and a reflux condenser were attached to a 1,000 ml four-neck flask and while carrying out deoxygenation by introduction of nitrogen gas, 350 ml of distilled water was charged therein and heated until the internal temperature reached 80° C. To the flask was added 1.5 g of sodium dodecylsufate as a dispersing agent, then was added 0.45 g of ammoniumpersulfate as an initiator, and thereafter was dropwise added 45.0 g of styrene through the dropping funnel over a period of about one hour. After the completion of the dropwise addition, the mixture was continued to react as it was for 5 hours, followed by removing the unreacted monomers by steam distillation. The mixture was cooled, adjusted the pH to 6 with aqueous ammonia and finally added pure water thereto so as to have the nonvolatile content of 15% by weight to obtain Aqueous dispersion of fine polymer particle (2). The particle size distribution of the fine polymer particle had the maximum value at the particle size of 60 nm.

The image-recording layer was prepared as described above, thereby preparing each of Lithographic printing plate precursors (34) to (36) for Examples 35 to 37.

### 5. Evaluation of Lithographic Printing Plate Precursor

### (1) Plate Inspection Property

Each of Lithographic printing plate precursors (1) to (37) and (39) to (42) thus-obtained was exposed by TRENDSETTER 3244VX (produced by Creo Co.) equipped with a water-cooled 40 W infrared semiconductor laser under the conditions of output of 11.7 W, a rotational number of an external drum of 250 rpm and resolution of 2,400 dpi. The exposed lithographic printing plate precursor was allowed to stand for 30 minutes in a dark place having atmosphere at 25° C. and relative humidity of 50% RH and then the plate inspection property was evaluated. Ease of plate inspection was measured using an L value (luminance) of L*a*b* color system and difference (ΔL) between an L value of the exposed area and an L value of the unexposed area was determined. When the value of ΔL is larger, it is meant that the plate inspection property is more excellent. The measurement was conducted according to SCE (specular competent exclude) system using a spectral colorimeter CM2600d and an operation soft CM-S100W each produced by Konica Minolta Holdings, Inc. According to the SCE system, since the specular light is excluded and only the diffusion light is measured, evaluation of color close to evaluation with visual observation is conducted and the result well correlates with the practical human plate inspection. The determinations of ΔL were conducted immediately after the exposure and at two hour lapse after the exposure kept at room temperature. The results obtained are shown in Tables 2 and 3.

### (2) On-Press Development Property

Each of the lithographic printing plate precursors thus-obtained was exposed by LUXEL PLATESETTER T-6000III equipped with an infrared semiconductor laser, produced by Fujifilm Corp. under the conditions of a rotational number of an external drum of 1,000 rpm, laser output of 70% and resolution of 2,400 dpi. The exposed image contained a solid image and a 50% halftone dot chart of a 20 μm-dot FM screen.

The exposed lithographic printing plate precursor was mounted without undergoing development processing on a plate cylinder of a printing machine (LITHRONE 26, produced by Komori Corp.). Using dampening water (ECOLITY-2 (produced by Fujifilm Corp.)/tap water=2/98 (volume ratio)) and VALUES-G (N) Black Ink (produced by Dainippon Ink & Chemicals, Inc.), the dampening water and ink were supplied according to the standard automatic printing start method of LITHRONE 26 to conduct printing on 100 sheets of TOKUBISHI ART PAPER (76.5 kg) at a printing speed of 10,000 sheets per hour.

A number of the printing papers required until the on-press development of the unexposed area of the image-recording layer on the printing machine was completed to reach a state where the ink was not transferred to the printing paper in the non-image area was measured to evaluate the on-press development property. The results obtained are shown in Tables 2 and 3.

### (3) Printing Durability

After performing the evaluation for the on-press development property described above, the printing was continued. As the increase in a number of printing papers, the image-recording layer was gradually abraded to cause decrease in the ink density on the printing paper. A number of the printing papers wherein a value obtained by measuring a halftone dot area rate of the 50% halftone dot of FM screen on the printing paper using a Gretag densitometer decreased by 5% from the value measured on the 100^{th} paper of the printing was determined to evaluate the printing durability. The results obtained are shown in Tables 2 and 3.

TABLE 2
Examples 2 to 31 and 38 and Comparative Examples 2 to 6

| | | | | | | Evaluation Result of Printing | | | |
|---|---|---|---|---|---|---|---|---|---|
| | | | | | | Plate | Plate | | |
| | | | | | | Inspection | Inspection | | |
| | Lithographic | Coating Solution | | | Coating | Property (ΔL) | Property (ΔL) | Printing | On-press |
| | Printing | for | | | Solution for | Immediately | Two Hours | Durability | Development |
| | Plate | Image-Recording | Specific | Radical | Protective | after | after | (×10⁴ | Property |
| | Precursor | Layer | Cyanine Dye | Generator | Layer | Exposure | Exposure | sheets) | (sheets) |
| Example 2 | (1) | (1) | A-1 | I-1 | (1) | 5.0 | 4.5 | 5 | 20 |
| Example 3 | (2) | (1) | A-2 | I-1 | (1) | 5.5 | 5.0 | 5 | 20 |
| Example 4 | (3) | (1) | A-3 | I-1 | (1) | 5.0 | 4.5 | 5 | 20 |
| Example 5 | (4) | (1) | A-4 | I-1 | (1) | 5.0 | 4.5 | 5 | 20 |
| Example 6 | (5) | (1) | A-5 | I-1 | (1) | 5.0 | 4.5 | 5 | 20 |
| Example 7 | (6) | (1) | A-6 | I-1 | (1) | 5.0 | 4.5 | 5 | 20 |
| Example 8 | (7) | (1) | A-7 | I-1 | (1) | 5.5 | 5.0 | 5 | 20 |
| Example 9 | (8) | (1) | A-11 | I-1 | (1) | 5.5 | 5.0 | 5 | 20 |
| Example 10 | (9) | (1) | A-12 | I-1 | (1) | 5.5 | 5.0 | 5 | 20 |
| Example 11 | (10) | (1) | A-13 | I-1 | (1) | 6.0 | 5.5 | 5 | 20 |
| Example 12 | (11) | (1) | A-19 | I-1 | (1) | 6.0 | 5.5 | 5 | 20 |
| Example 13 | (12) | (1) | A-23 | I-1 | (1) | 5.0 | 4.5 | 5 | 20 |
| Example 14 | (13) | (1) | A-24 | I-1 | (1) | 4.5 | 4.0 | 5 | 20 |
| Example 15 | (14) | (1) | A-25 | I-1 | (1) | 4.5 | 4.0 | 5 | 20 |
| Example 16 | (15) | (1) | A-26 | I-1 | (1) | 4.5 | 4.0 | 5 | 20 |
| Example 17 | (16) | (1) | A-27 | I-1 | (1) | 4.5 | 4.0 | 5 | 20 |
| Example 18 | (17) | (1) | A-28 | I-1 | (1) | 4.5 | 4.0 | 5 | 20 |
| Example 19 | (18) | (1) | A-31 | I-1 | (1) | 4.5 | 4.0 | 5 | 20 |
| Example 20 | (19) | (1) | A-32 | I-1 | (1) | 5.5 | 5.0 | 5 | 20 |
| Example 21 | (20) | (1) | A-33 | I-1 | (1) | 5.5 | 5.0 | 5 | 20 |
| Example 22 | (21) | (1) | A-34 | I-1 | (1) | 5.5 | 5.0 | 5 | 20 |
| Example 23 | (22) | (1) | A-37 | I-1 | (1) | 6.0 | 5.5 | 5 | 20 |
| Example 24 | (23) | (1) | A-43 | I-1 | (1) | 4.5 | 3.0 | 5 | 20 |
| Example 25 | (24) | (1) | A-44 | I-1 | (1) | 5.5 | 4.5 | 5 | 20 |
| Example 26 | (25) | (1) | A-45 | I-1 | (1) | 5.0 | 4.5 | 5 | 20 |
| Example 27 | (26) | (1) | A-46 | I-1 | (1) | 5.0 | 4.5 | 5 | 20 |
| Example 28 | (27) | (1) | A-47 | I-1 | (1) | 4.5 | 4.0 | 5 | 20 |
| Example 29 | (28) | (1) | A-48 | I-1 | (1) | 4.5 | 4.0 | 5 | 20 |
| Example 30 | (29) | (1) | A-2 | I-2 | (1) | 5.0 | 4.5 | 4 | 20 |
| Example 31 | (30) | (1) | A-2 | I-3 | (1) | 5.0 | 4.5 | 4 | 20 |
| Example 38 | (37) | (1) | A-2 | I-1 | (2) | 5.0 | 4.5 | 3 | 30 |
| Comparative | (39) | (1) | A-49 | I-1 | (1) | 4.0 | 1.5 | 5 | 20 |
| Example 2 | | | | | | | | | |
| Comparative | (40) | (1) | A-50 | I-1 | (1) | 4.0 | 1.5 | 5 | 20 |
| Example 3 | | | | | | | | | |
| Comparative | (41) | (1) | A-51 | I-1 | (1) | 4.0 | 1.5 | 5 | 20 |
| Example 4 | | | | | | | | | |
| Comparative | (42) | (1) | A-52 | I-1 | (1) | 3.5 | 1.0 | 5 | 20 |
| Example 5 | | | | | | | | | |
| Comparative | (43) | (1) | A-50 | I-1 | (2) | 3.5 | 1.0 | 3 | 30 |
| Example 6 | | | | | | | | | |

TABLE 3
Examples 32 to 37 and Comparative Example 7

| | | | | | | Evaluation Result of Printing | | | |
|---|---|---|---|---|---|---|---|---|---|
| | | | | | | Plate | Plate | | |
| | | | | | | Inspection | Inspection | | |
| | Lithographic | Coating Solution | | | Coating | Property (ΔL) | Property (ΔL) | Printing | On-press |
| | Printing | for | | | Solution for | Immediately | Two Hours | Durability | Development |
| | Plate | Image-Recording | Specific | Radical | Protective | after | after | (×10⁴ | Property |
| | Precursor | Layer | Cyanine Dye | Generator | Layer | Exposure | Exposure | sheets) | (sheets) |
| Example 32 | (31) | (2) | A-2 | Irg. 250 | None | 5.0 | 4.0 | 3 | 30 |
| Example 33 | (32) | (2) | A-13 | Irg. 250 | None | 5.5 | 4.5 | 3 | 30 |
| Example 34 | (33) | (2) | A-27 | Irg. 250 | None | 4.5 | 3.5 | 3 | 30 |
| Example 35 | (34) | (3) | A-2 | I-1 | None | 5.0 | 4.0 | 3 | 30 |
| Example 36 | (35) | (3) | A-13 | I-1 | None | 5.5 | 4.5 | 3 | 30 |
| Example 37 | (36) | (3) | A-27 | I-1 | None | 4.5 | 3.5 | 3 | 30 |
| Comparative | (44) | (2) | A-50 | Irg. 250 | None | 3.5 | 1.0 | 3 | 30 |
| Example 7 | | | | | | | | | |

In Tables 2 and 3, the symbols in the columns of the specific cyanine dye indicate the numbers of the compounds in the specific examples described hereinbefore, respectively, and Cyanine dyes A-49 to A-52 used for comparison are shown below.

Further, in the columns of the radical initiator of Tables 2 and 3, I-1 is same compound as used in Example 1 and I-2 to I-3 indicate the compounds having the structure shown below, respectively. Also, “Irg. 250” indicates IRGACURE 250.

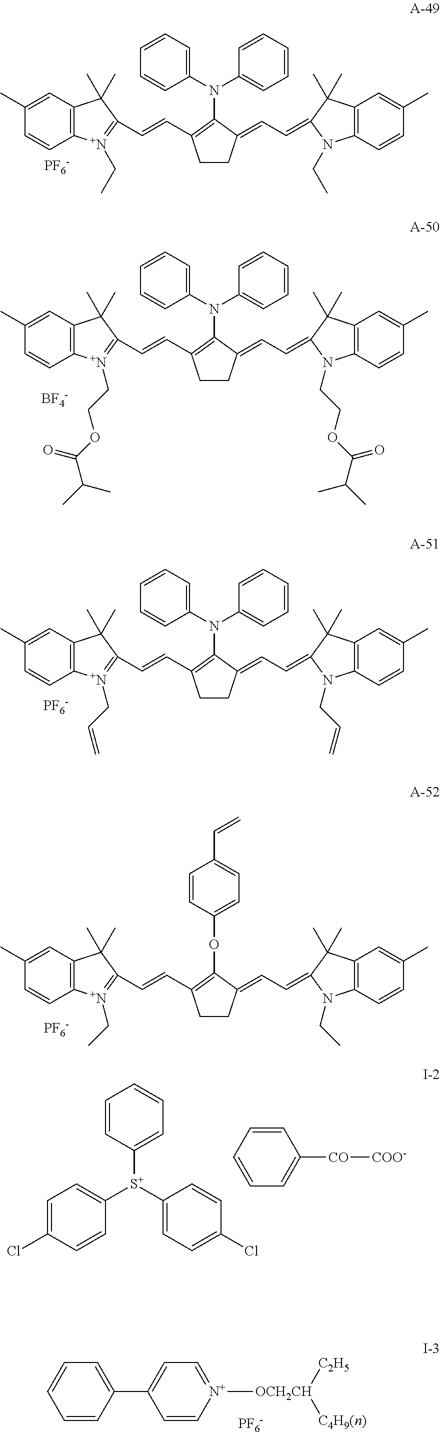

From the results shown above, it can be seen that the good color image is obtained after image exposure and the good color image is maintained with the lapse of time according to the invention. Further, the on-press development property and printing durability are also good.

What is claimed is:

1. A color-forming photosensitive composition comprising:
a cyanine dye having at least two polymerizable groups selected from an acryloyl group, a methacryloyl group and a vinyl group;
a radical generator; and
a monomer having an ethylenically unsaturated group.

2. The color-forming photosensitive composition as claimed in claim 1, wherein the cyanine dye is represented by the following formula (1): 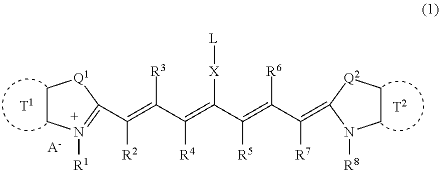
wherein R¹ and R⁸ represent identical organic groups having a polymerizable group selected from an acryloyl group, a methacryloyl group and a vinyl group, R², R³, R⁴, R⁵, R⁶ and R⁷ each independently represents a hydrogen atom or a hydrocarbon group, R⁴ and R⁵ may be combined with each other to form a ring, X represents an oxygen atom, a nitrogen atom or a sulfur atom, L represents an aromatic ring group, a heteroaromatic ring group or an alkyl group having from 1 to 12 carbon atoms which may contain a hetero atom, provided that when X represents a nitrogen atom, X-L represents —N(L1)(L2) wherein L1 and L2, which may be the same or different, each represents the same group as defined for L, Q¹ and Q², which may be the same or different, each represents —NR⁹—, a sulfur atom, an oxygen atom or a dialkylmethylene group, R⁹ represents a hydrogen atom or a hydrocarbon group which may have a substituent, T¹ and T² each independently represents an aromatic ring which may further have a substituent or a heteroaromatic ring which may further have a substituent, and A⁻ represents a counter ion.

3. The color-forming photosensitive composition as claimed in claim 1, wherein the cyanine dye is represented by the following formula (2): 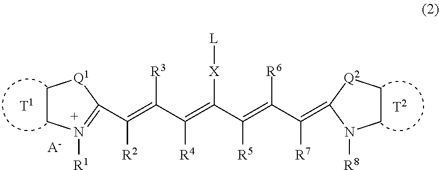
wherein R¹, R², R³, R⁴, R⁵, R⁶, R⁷ and R⁸ each independently represents a hydrogen atom or a hydrocarbon group, R⁴ and R⁵ may be combined with each other to form a ring, X represents an oxygen atom, a nitrogen atom or a sulfur atom, L represents an aromatic ring group, a heteroaromatic ring group or an alkyl group having from 1 to 12 carbon atoms which may contain a hetero atom, provided that when X represents a nitrogen atom, X-L represents —N(L1)(L2) wherein L1 and L2, which may be the same or different, each represents the same group as defined for L, Q¹ and Q², which may be the same or different, each represents —NR⁹—, a sulfur atom, an oxygen atom or a dialkylmethylene group, R⁹ represents a hydrogen atom or a hydrocarbon group which may have a substituent, T¹ and T² represent the identical aromatic rings or heteroaromatic rings comprising an organic group having a polymerizable group selected from an acryloyl group, a methacryloyl group and a vinyl group, and A⁻ represents a counter ion.

4. The color-forming photosensitive composition as claimed in claim 1, wherein the cyanine dye is represented by the following formula (3): 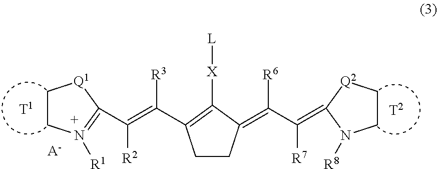
wherein R¹ and R⁸ represent identical organic groups having a polymerizable group selected from an acryloyl group, a methacryloyl group and a vinyl group, R², R³, R⁶ and R⁷ each independently represents a hydrogen atom or a hydrocarbon group, X represents an oxygen atom, a nitrogen atom or a sulfur atom, L represents an aromatic ring group, a heteroaromatic ring group or an alkyl group having from 1 to 12 carbon atoms which may contain a hetero atom, provided that when X represents a nitrogen atom, X-L represents —N(L1)(L2) wherein L1 and L2, which may be the same or different, each represents the same group as defined for L, Q¹ and Q², which may be the same or different, each represents —NR⁹—, a sulfur atom, an oxygen atom or a dialkylmethylene group, R⁹ represents a hydrogen atom or a hydrocarbon group which may have a substituent, T¹ and T² each independently represents an aromatic ring which may further have a substituent or a heteroaromatic ring which may further have a substituent, and A⁻ represents a counter ion.

5. The color-forming photosensitive composition as claimed in claim 1, wherein the radical generator is an iodonium salt, a sulfonium salt or an azinium salt.

6. The color-forming photosensitive composition as claimed in claim 1, which further comprises a binder polymer.

7. A lithographic printing plate precursor comprising a support and an image-recording layer comprising the color-forming photosensitive composition as claimed in claim 1.

8. The lithographic printing plate precursor as claimed in claim 7, wherein the image-recording layer further comprises a hydrophobilizing precursor.

9. The lithographic printing plate precursor as claimed in claim 7, which further comprises a protective layer so that the support, the image-recording layer and the protective layer are provided in this order.

10. The lithographic printing plate precursor as claimed in claim 9, wherein the protective layer comprises an inorganic stratiform compound.

11. A method for forming a color image, comprising: exposing imagewise the color-forming photosensitive composition as claimed in claim 1 to form color in the exposed area.

12. A plate making method comprising:
exposing imagewise the lithographic printing plate precursor as claimed in claim 7 to form color in the exposed area and mounting the exposed lithographic printing plate precursor on a printing machine, or mounting the lithographic printing plate precursor as claimed in claim 7 on a printing machine and exposing imagewise the mounted lithographic printing plate precursor to form color in the exposed area; and
conducting on-press development processing by supplying printing ink and dampening water to the exposed and mounted lithographic printing plate precursor.

13. A cyanine dye represented by the following formula (4): 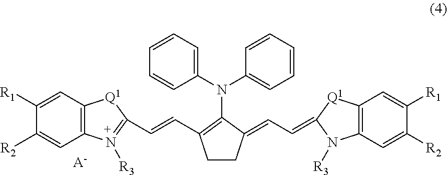
wherein R₁ and R₂ each independently represents a hydrogen atom, a methyl group, a methoxy group or a halogen atom, or R₁ and R₂ may be combined with each other to form a naphtho condensed ring, Q¹ represents a sulfur atom or a dialkylmethylene group, A⁻ represents a halide ion, a perchlorate ion, a tetrafluoroborate ion, a hexafluorophosphate ion or a sulfonate ion, and R₃ represents an organic group having an acryloyl group or a methacryloyl group.

14. The cyanine dye as claimed in claim 13, wherein, in the formula (4), when Q¹ represents a sulfur atom, R₁ represents a hydrogen atom and R₂ represents a hydrogen atom, a methyl group, a methoxy group or a halogen atom, or when Q¹ represents a dimethylmethylene group, R₂ represents a hydrogen atom and R₁ represents a hydrogen atom, a methyl group, a methoxy group or a halogen atom, A⁻ represents a hexafluorophosphate ion, and R₃ represents an organic group having an acryloyl group or a methacryloyl group.
